(19) United States
(12) Patent Application Publication
(10) Pub. No.: US 2026/0284296 A1
(43) Pub. Date: Sep. 24, 2026

(54) TRANSDERMAL DRUG DELIVERY DEVICES AND METHODS

(71) Applicant: MORNINGSIDE VENTURE INVESTMENTS LIMITED, Newton Centre, MA (US)

(72) Inventors: Michael P. SCHALLER, Louisville, CO (US); Andrew L. JOHNSTON, Redwood City, CA (US); Patrick H. RUANE, Dublin, CA (US); Carolyn G. STONE, Los Gatos, CA (US); Julianna K. SWARTZENBERG, Spring, TX (US); Kevin W. GELSTON, Moraga, CA (US); Luke W. CLAUSON, Redwood City, CA (US)

(21) Appl. No.: 19/561,324

(22) Filed: Mar. 9, 2026

Related U.S. Application Data
(62) Division of application No. 16/462,971, filed on Dec. 5, 2017, now abandoned.
(60) Provisional application No. 62/430,121, filed on Dec. 5, 2016.
(60) Provisional application No. 62/443,526, filed on Jan. 6, 2017.

(51) Int. Cl. A61M 5/00; A61K 9/00; A61K 9/70; A61M 31/00; A61M 35/00

(52) U.S. Cl. CPC A61M 5/00; A61K 9/0014; A61K 9/7084; A61K 9/7092; A61M 31/002; A61M 35/10; A61M 2205/50; A61M 2205/8206

(57) ABSTRACT

A system for delivering a drug formulation includes a transdermal membrane, a drug cartridge, and a control unit having a battery, a printed circuit board for actuation of the actuator. The drug cartridge includes a sealed reservoir configured to hold a drug formulation therein, a breaking element configured to break the sealed reservoir upon actuation, and an actuator configured to activate the breaking element to break the sealed reservoir release the drug formulation to the transdermal membrane. The control unit can include a controller configured to activate the actuator at a preset time.

### CROSS REFERENCE TO RELATED APPLICATIONS

This application is a divisional of U.S. application Ser. No. 16/462,971 filed May 22, 2019, which is a 371 of International Application No. PCT/ 2017/064765 filed Dec. 5, 2017 which claims priority to U.S. Provisional Application No. 62/430,121, filed Dec. 5, 2016, entitled “MORNING ONE SHOT”, and U.S. Provisional Application No. 62/443,526, filed Jan. 6, 2017, entitled “TRANSDERMAL DRUG DELIVERY DEVICES AND METHODS”, the entireties of which are incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present application relates generally to devices and methods for transdermally providing a bioactive agent or drug formulation to a user.

### BACKGROUND

Tobacco use, such as smoking, causes serious health problems and can lead to premature death. According to the United States Center for Disease Control (CDC), tobacco use causes more than 5 million deaths per year as well as contributes to the development of serious illnesses such as cancer, diabetes, heart disease, lung disease (bronchitis, chronic airway destruction, emphysema), and stroke. Despite anti-smoking advertising campaigns, legislation, taxation, and development of smoking cessation products to stop or prevent people from using tobacco, tobacco sales remains a multibillion dollar industry, generating an estimated $35 billion dollars per year in profits. Tobacco initially causes physical and mood-altering effects that are temporarily pleasing. Further, it is difficult for a person to stop using a tobacco product because tobacco contains nicotine. Nicotine is highly addictive, and not having the nicotine can cause harsh withdrawal symptoms. It can be very difficult for a person to overcome nicotine addiction and stop smoking.

Medicinal drugs can be taken by tobacco users to help overcome their nicotine addiction and stop using tobacco. Some products to help a person stop smoking contain small amounts of nicotine to minimize withdrawal symptoms and gradually wean a person from their nicotine addiction. Medicinal smoking cessation drugs such as nicotine generally have to be taken over an extended period of time (often over the course of many months) to give the body time to adjust to having less nicotine. Medicinal drugs, medical devices and other products, including smoking cessation products, are regulated in the United States by the U.S. Food and Drug Administration (FDA). FDA approved products on the market to help a person quit smoking include various medicinal drugs that require a doctor's prescription as well as over-the-counter products. These products include capsules or tablets, gums, inhalers, lozenges, nasal sprays, and skin patches. These products, however, have thus far been inadequate to get people to stop smoking Indeed, 68.9 % of adult cigarette smokers say they want to stop smoking, and every year some 42.7% make an attempt to stop smoking, but are unsuccessful.

Existing smoking cessation products and other therapeutic and prophylactic treatments for health issues suffer from a variety of problems. They may be inconvenient or socially awkward to use. They may require careful and troublesome tracking of when they were used and how much was used to prevent overdosing. They may act too slowly after being administered and not produce a desired effect when it's needed. They may not be readily available when they are needed, such as while a person is sleeping. None have been wholly effective for preventing or treating various medical or other conditions.

One problem with smoking cessation products is that patients tend to wake up in the morning, and any cessation drug from the previous day has been already metabolized and cleared from their body, resulting in severe morning symptoms or cravings immediately upon waking. Current therapies may deliver drug during sleep, but doing so can cause sleep disturbances and is often not optimized to ensure high amounts upon waking with minimal side effects. In short, current therapies do not have the capability to provide the tailored drug delivery profile needed to optimize therapy and decrease adverse side effects. As a result, patients must either endure adverse symptoms upon waking up until the morning dose of medication reaches therapeutically effective amounts or disrupt their sleep to wake up early and dose themselves. Accordingly, there is a critical unmet need for an automated therapeutically effective bolus at a preset time (e.g., pre-wake-up morning bolus to pre-emptively offset upon wake-up morning symptoms). Such a system could ensure therapeutically effective amounts of drug are present in plasma immediately prior to wake up, while ensuring minimal amount of drug is delivered during sleep.

Additionally, some medicinal drugs, e.g., for smoking cessation, are rapidly metabolized by the body. Multiple doses of the drug over a period of time are thus often needed to provide a desired effect. A drug delivery system that provides such multiple doses is therefore also desired.

### SUMMARY OF THE DISCLOSURE

The present disclosure relates generally to apparatuses, systems and methods for delivering medicines, drug formulations, and/or other bioactive agents.

In general, in one embodiment, a system for delivering a drug formulation includes a transdermal membrane, a drug cartridge, a control unit having a battery and a printed circuit board for actuation of the actuator. The cartridge includes a sealed reservoir, a breaking element, an actuator, and a pressurizing element. The drug reservoir is configured to hold a drug formulation therein. The breaking element is configured to break the sealed reservoir upon actuation. The actuator is configured to activate the breaking element to break the sealed reservoir. The pressurizing element is configured to place pressure on the sealed reservoir such that, upon activation of the breaking element, drug formulation from the sealed reservoir is forced out of the sealed reservoir and onto the transdermal membrane.

This and other embodiments can include one or more of the following features. The pressurizing element can be a compressed material. The compressed material can be foam. The pressurizing element can be a plunger. The pressurizing element can be a leaf spring. The breaking element can be a blade. The breaking element can be a needle. The breaking element can be a SMA wire embedded in a portion of the reservoir. A wall of the reservoir can be dissolvable, and the breaking element can be a solvent configured to dissolve the wall. A wall of the reservoir can be meltable, and the breaking element can be a heater configured to melt the wall. The cartridge can include a second sealed reservoir configured to hold a drug formulation therein. The cartridge can include a second breaking element configured to break the second sealed reservoir upon activation. The system can be configured to release drug formulation from the two reservoirs at different times. The pressurizing element can be on a side of the reservoir that is opposite of the transdermal membrane. The breaking element can be configured to pierce the reservoir from a side of the reservoir that is closer to the transdermal membrane than the pressurizing element. The actuator can be a fuse wire. The actuator can be a shape memory alloy wire. The actuator can be a motor and cam. The system can have no motor. The control unit can include a controller configured to activate the actuator at a preset time. The cartridge can be disposable and the control unit can be reusable. The drug cartridge and the control unit can be separable from one another. The breaking element can be a flat plate configured to crush the reservoir.

In general, in one embodiment, a system for delivering a drug formulation includes a transdermal membrane, a drug cartridge, and a control unit having a battery and a printed circuit board for actuation of the actuator. The drug cartridge includes a sealed reservoir, a breaking element, an actuator, and an expandable element. The sealed reservoir is configured to hold a drug formulation therein. The breaking element is configured to break the sealed reservoir upon actuation. The expandable element is connected to the breaking element. The actuator is configured to activate the expandable element. Upon actuation by the actuator, the expandable element expands to cause the breaking element to break the sealed reservoir and release drug formulation to the transdermal membrane.

This and other embodiments can include one or more of the following features. The expandable element can be a pre-compressed spring. The expandable element can be a heat expansive element. The cartridge can be disposable and the control unit can be reusable. The drug cartridge and the control unit can be separable from one another. The breaking element can be a blade. The breaking element can be a needle. The breaking element can be a SMA wire embedded in a portion of the reservoir. A wall of the reservoir can be dissolvable, and the breaking element can be a solvent that can be configured to dissolve the wall. A wall of the reservoir can be meltable, and the breaking element can be a heater that can be configured to melt the wall. The system can further include a second sealed reservoir that can be configured to hold a drug formulation therein. The system can further include a second breaking element that can be configured to break the second sealed reservoir upon activation. The system can be configured to release drug formulation from the two reservoirs at different times. The control unit can include a controller that can be configured to activate the actuator at a preset time. The breaking element can be configured to pierce the reservoir from a side of the reservoir that is closest to the transdermal membrane. The actuator can include a fuse wire. The actuator can be a shape memory alloy wire. The actuator can be a motor and cam. The system can have no motor. The breaking element can be a flat plate that can be configured to crush the reservoir.

In general, in one embodiment, a system for delivering a drug formulation includes a transdermal membrane, a drug cartridge and a control unit having a battery and a controller. The controller can be configured to activate the actuator at a pre-set time. The drug cartridge can include a sealed reservoir configured to hold a drug formulation therein, a breaking element configured to break the sealed reservoir upon actuation, and an actuator configured to activate the breaking element to break the sealed reservoir and release drug formulation to the transdermal membrane.

This and other embodiments can include one or more of the following features. The control unit can further include a user interface configured to allow a patient to set the pre-set time for actuation of the actuator and thus release of the drug formulation. The pre-set time can be prior to a wake-up time of a patient wearing the system. The pre-set time can be programmed by a device paired wirelessly with the system. The device can be a smart phone or computer. The breaking element can be a blade. The breaking element can be a needle. The breaking element can be a SMA wire embedded in a portion of the reservoir. A wall of the reservoir can be dissolvable, and the breaking element can be a solvent that can be configured to dissolve the wall. The system can further comprise a second sealed reservoir configured to hold a drug formulation therein. The system can further comprise a second breaking element that can be configured to break the second sealed reservoir upon activation. The controller can be configured to release drug formulation from the two reservoirs at two different preset times. The drug cartridge and the control unit can be separable from one another. The breaking element can be configured to pierce the reservoir from a side of the reservoir that is closest to the transdermal membrane. The actuator can include a fuse wire. The actuator can be a shape memory alloy wire. The actuator can be a motor and cam. The system can have no motor. The cartridge can be disposable and the control unit can be reusable.

In general, in one embodiment, a method for delivering a drug formulation includes: placing a drug cartridge in contact with a skin of a patient, where the drug cartridge comprises a sealed reservoir containing drug formulation; and setting a preset time for release of the drug formulation to the patient's skin such that, at the preset time, a breaking element is activated to break the sealed reservoir such that drug formulation flows from the reservoir to the patient's skin.

This and other embodiments can include one or more of the following features. The pre-set time can be while the patient is expected to be sleeping just prior to wake-up. Placing the drug cartridge can occur before the patient goes to sleep.

In general, in one embodiment, a system for delivering a drug formulation includes a transdermal membrane, a drug cartridge, and a control unit having a battery, a printed circuit board for actuation of the actuator. The drug cartridge includes a sealed reservoir configured to hold a drug formulation therein, a breaking element configured to break the sealed reservoir upon actuation, and an actuator configured to activate the breaking element to break the sealed reservoir release the drug formulation to the transdermal membrane.

This and other embodiments can include one or more of the following features. The system can further include a pressurizing element configured to place pressure on the sealed reservoir such that, upon activation of the breaking element, drug formulation from the sealed reservoir that can be forced out of the sealed reservoir and onto the transdermal membrane. The system can further include an expandable element connected to the breaking element such that, upon actuation by the actuator, the expandable element expands to cause the breaking element to break the sealed reservoir and release drug formulation to the transdermal membrane. The cartridge can be disposable and the control unit is reusable. The drug cartridge and the control unit can be separable from one another. The breaking element can be a blade. The breaking element can be a needle. The breaking element can be an SMA wire embedded in a portion of the reservoir. A wall of the reservoir can be dissolvable, and the breaking element can be a solvent configured to dissolve the wall. A wall of the reservoir can be meltable, and the breaking element can be a heater configured to melt the wall. The system can further include a second sealed reservoir configured to hold a drug formulation therein. The system can further include a second breaking element that can be configured to break the second sealed reservoir upon activation. The system can be configured to release drug formulation from the two reservoirs at different times. The breaking element can be configured to pierce the reservoir from a side of the reservoir that can be closest to the transdermal membrane. The actuator can further include a fuse wire. The actuator can be a shape memory alloy wire. The actuator can be a motor and cam. The system can have no motor. The control unit can include a controller configured to activate the actuator at a preset time.

In general, in one embodiment, a system for delivering a drug formulation comprises a transdermal membrane and a drug cartridge. The drug cartridge can include a sealed reservoir that is configured to hold a drug formulation therein, and a barrier that prevents drug formulation from being released from the sealed reservoir, and an actuator configured to move the barrier such that drug formulation can be released to the transdermal membrane.

This and other embodiments can include one or more of the following features. The barrier can form a wall of the sealed reservoir. The barrier can include a plurality of panes configured to sequentially rotate to release fluid from the reservoir. The barrier can include a plurality of pores configured to open upon activation. The system can further include an outlet extending from the sealed reservoir to the transdermal membrane. The system can further include a delivery port extending from the sealed reservoir to the transdermal membrane, the barrier can extend between the drug cartridge and the delivery port. The actuator can be a resistance wire configured to expand to push the barrier away from the port. The actuator can be a resistance wire that can be configured to burn to release the barrier away from the port. The barrier can be configured to melt upon activation.

In general, in one embodiment, a system for delivering bioactive agents includes a drug cartridge (DC). The drug cartridge includes one or more sealed reservoirs configured to contain drug formulation and one or more outlet ports configured to guide the drug formulation onto a membrane in contact with a skin of a patient. Each of the one or more outlet ports is disposed under each of the one or more sealed reservoirs. The drug cartridge further includes one or more sliding shuttles, each of the one or more sliding shuttles comprises a spring-loaded sharp-edge component for puncturing each of the one or more sealed reservoirs upon actuation, and one or more actuators configured to activate the one or more sliding shuttles to puncture the one or more sealed reservoirs to enable the drug formulation to be released and flow into the membrane. The system further includes a Control Unit (CU) comprises a holder to hold a battery and a printed circuit board.

This and other embodiments can include one or more of the following features. The system can further include one or more latches configured to hold the one or more sliding shuttles in place until the time of actuation. The one or more actuators can comprise fuse wires. The length, width, and thickness of the system are between 40 mm-100 mm, 10 mm-70 mm, and 1 to 20 mm, respectively. The one or more actuators can comprise Shape-memory alloy (SMA) wires. The system can include one or more posts. The length, width, and thickness of the system can be between 30 mm-100 mm, 10 mm-70 mm, and 1 mm to 15 mm, respectively. The DC can be disposable and the CU can be reusable. The system can have no motor. The system can further include a compressible material or a heat expandable material that are configured to further enable the one or more reservoirs to empty.

In general, in one embodiment, an apparatus for delivering bioactive agents comprises a drug cartridge (DC). The drug cartridge includes one or more sealed reservoirs, one or more outlet ports, one or more sliding shuttles, and one or more actuators. The one or more reservoirs contain drug formulation. The one or more outlet ports are configured to guide the drug formulation onto a membrane in contact with a skin of a patient. Each of the one or more outlet ports are disposed under each of the one or more reservoirs. Each of the one or more sliding shuttles further include a spring-loaded sharp-edge component for puncturing each of the one or more reservoirs upon actuation. The one or more actuators can be configured to activate the one or more sliding shuttles to puncture the one or more sealed reservoirs and enable the drug formulation to be released and flow into the membrane.

The apparatus can further include one or more latches that can be configured to hold the one or more sliding shuttles in place until the time of actuation. The one or more actuators can include fuse wires. The length, width, and thickness of the apparatus can be between 40 mm-100 mm, 10 mm-70 mm, and 1 to 20 mm, respectively. The one or more actuators can include Shape-memory alloy (SMA) wires. The apparatus can further include one or more posts. The length, width, and thickness of the system can be between 30 mm-100 mm, 10 mm-70 mm, and 1 mm to 20 mm, respectively. The DC can be disposable. The apparatus can have no motor. The apparatus can further comprise a foam configured to further enable the one or more reservoirs to empty.

In general, in one embodiment, a method for delivering bioactive agents includes placing a drug cartridge (DC) in contact with a skin of a patient. The DC includes one or more sealed reservoirs containing drug formulation and one or more outlet ports configured to guide the drug formulation onto a membrane. The method further includes activating one or more sliding shuttles. Each of the one or more sliding shuttles includes a spring-loaded sharp-edge component that punctures each of the one or more sealed reservoirs when actuated by the actuator. This enables the drug formulation from the one or more sealed reservoirs to be released and flow into the membrane.

This and other embodiments can include one or more of the following features. The method can further release one or more latches, where the one or more latches are configured to hold the one or more sliding shuttles in place until the time of actuation. The one or more actuators can include fuse wires. The one or more actuators can include Shape-memory alloy (SMA) wires. The method can include activating without using a motor.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows an exemplary transdermal drug delivery device.

FIGS. 2A-2F shows an exemplary two-part transdermal drug delivery device.

FIGS. 3A-3D show an exemplary transdermal drug delivery device with a multi-reservoir cartridge.

FIGS. 4A-4D show an exemplary transdermal drug delivery device with a multi-reservoir cartridge having a shape memory alloy wire actuator release mechanism.

FIG. 5 shows an exemplary transdermal drug delivery device in a circular shape.

FIG. 6 shows an exemplary transdermal drug delivery device in a rectangular shape with rounded corners.

FIGS. 7A-7C show an exemplary transdermal drug delivery device configured to release formulation upon alignment of holes in the reservoir and the barrier.

FIGS. 8A-8C show an exemplary transdermal drug delivery device where a reservoir and a barrier are rotated relative to one another with a crank mechanism.

FIGS. 9A-9B show an exemplary transdermal drug delivery device wherein a reservoir and a barrier are rotated relative to one another with a pulley system.

FIGS. 10A-10B show a wire for use with a pulley system similar to that of FIGS. 9A-9B where the wire is pulled straight back (FIG. 10A) or wrapped around (FIG. 10B).

FIGS. 11A-11B show an exemplary transdermal drug delivery device with a windmill-type design.

FIG. 12 shows an exemplary transdermal drug delivery device with a needle configured to pierce a drug reservoir.

FIGS. 13A-13B show an exemplary transdermal drug delivery device with a meltable drug reservoir.

FIGS. 14A-14C show an exemplary transdermal drug delivery device configured as a gel patch.

FIGS. 15A-15B show an exemplary transdermal drug delivery device with a pod holding the drug formulation and a solvent configured to dissolve the pod.

FIGS. 16A-16C show an exemplary transdermal drug delivery device with a drug reservoir and a barrier blocking release of fluid therefrom.

FIGS. 17A-17B show an exemplary transdermal drug delivery device with a membrane having expandable pores.

FIG. 18 shows an exemplary transdermal drug delivery device with a blade configured to cut a seal leading to the drug reservoir.

FIGS. 19A-19B show an exemplary transdermal drug delivery device similar to FIG. 18 where the blade is activated by a cam.

FIGS. 20A-20D show an exemplary transdermal drug delivery device similar to FIG. 18 where the blade is activated by a compressed spring.

FIGS. 21A-21C show an exemplary transdermal drug delivery device with a drug cartridge made of a plurality of layers.

FIGS. 22A-22D show an exemplary transdermal drug delivery device with a reservoir that is configured to burst under pressure.

FIGS. 23A-23B show an exemplary transdermal drug delivery device including a barrier between the drug reservoir and the outlet.

FIGS. 24A-24B show another exemplary transdermal drug delivery device including a barrier between the drug reservoir and the outlet.

FIGS. 25A-25B show an exemplary transdermal drug delivery device including a meltable barrier between the drug reservoir and the transdermal membrane.

FIG. 26 shows an exemplary transdermal drug delivery device including a solvent reservoir and a drug reservoir.

FIG. 27 shows an exemplary transdermal drug delivery device including a layer of dehydrated drug and a solvent reservoir.

FIG. 28 shows an exemplary transdermal drug delivery device with a microblower configured to deliver the drug formulation.

FIGS. 29A-29C show an exemplary transdermal drug delivery device with a heat-expansive membrane that can force drug out of the drug reservoir.

FIGS. 30A-30B show an exemplary transdermal drug delivery device with a drug reservoir surrounded by a thin burst casing.

FIGS. 31A-31D show an exemplary transdermal drug delivery device with a wire configured to puncture a hole in a barrier layer of a drug reservoir.

FIGS. 32A-32D show the drug delivery device of FIGS. 31A-31D with the coil in an activated configuration.

FIGS. 33A-33B show an exemplary transdermal drug delivery device with a plunger configured to break the drug reservoir.

### DETAILED DESCRIPTION

Apparatuses, systems and methods are disclosed herein for transdermal delivery of a medicine. In particular, described herein is small, automated passive diffusion transdermal drug delivery device. The devices can be programmed with a preset delivery time.

In some embodiments, the transdermal drug devices described herein can include a formulation reservoir that is pierced or otherwise broken by a breaking element. The breaking elements can be, for example, a blade, a needle, an SMA wire embedded into the material of the reservoir chamber, or a solvent dissolving reservoir bag material. Actuation of the device can lead to piercing/rupture of formulation reservoir and delivery of the formulation onto a skin-contacting membrane of the device. Further, an element can push the piercing element forward during actuation, such as a compressed spring or a heat expandable element.

In some embodiments, the transdermal drug delivery devices described herein can include a formulation reservoir having one side formed or blocked by a barrier that keeps the formulation from entering delivery ports of the device. Actuation of the device can lead to the motion of the barrier and thus release of the formulation towards a skin-contacting membrane of the device.

In some embodiments, the transdermal drug delivery devices described herein can include a formulation reservoir with one side formed or blocked by a barrier that keeps the formulation from contacting the skin or skin-contacting membrane. Actuation of the device can lead to the motion of barrier and exposure of drug formulation to skin or to skin-contacting membrane/surface.

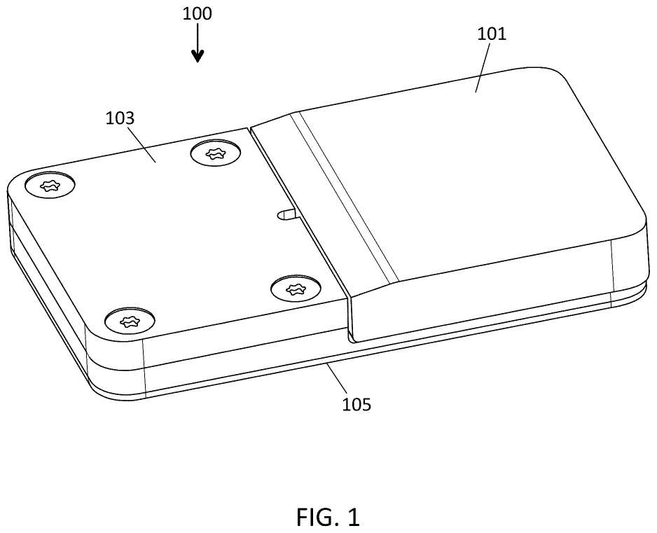

Referring to FIG. 1, a transdermal drug delivery device 100 can include a drug reservoir 103, an adhesive interface 105, and a programmable digital user interface 101. The programmable digital user interface 101 can be configured such that the patient can program in the anticipated wake-up time. The device can be placed on a patient's body/skin, e.g., before retiring for bed. The pre-programmed device 100 can automatically activate or turn on at an appropriate period of time before the set patient wake-up time. Once activated and while the patient is asleep, the device 100 can deliver one or more drug boluses from the drug reservoir 103 to the adhesive interface 105 for delivery to the patient's skin. By delivering the bolus a set time before the pre-programmed wake-up time, the device 100 advantageously ensures that a therapeutically effective amount of drug is present in the patient's plasma immediately upon the patient's wake-up in the morning. Such a time bolus can help offset morning symptoms or cravings. The device 100, when delivering a bolus of drug before wake-up, can be called a “morning one-shot” device.

Thus, the device 100 can advantageously deliver drug formulation to the patient at night or in the early morning while the patient is still sleeping so that the patient can be effectively medicated upon waking up. Use of the device 100 can automatically offset the manifestation of peak morning symptoms that are well known to accompany a wide array of diseases and addictions.

The device 100 can be small and comfortable so as to not interrupt the user's sleep. Further, because the drug is delivered automatically through a passive transdermal route, the patient need not be disturbed and yet still receives the effective dosage before waking.

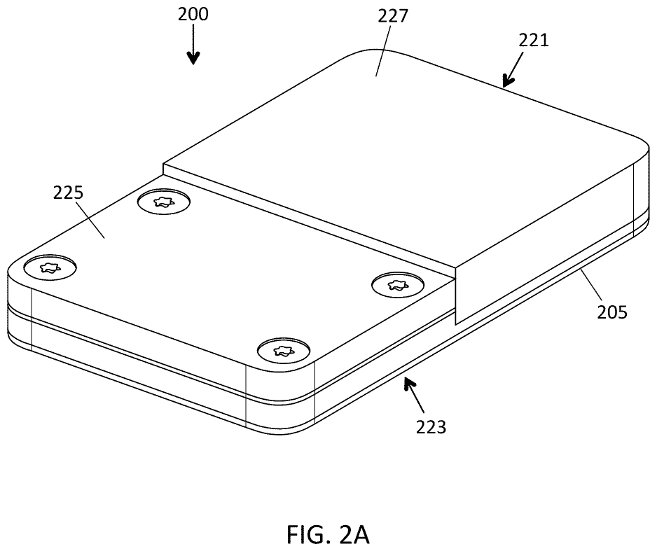

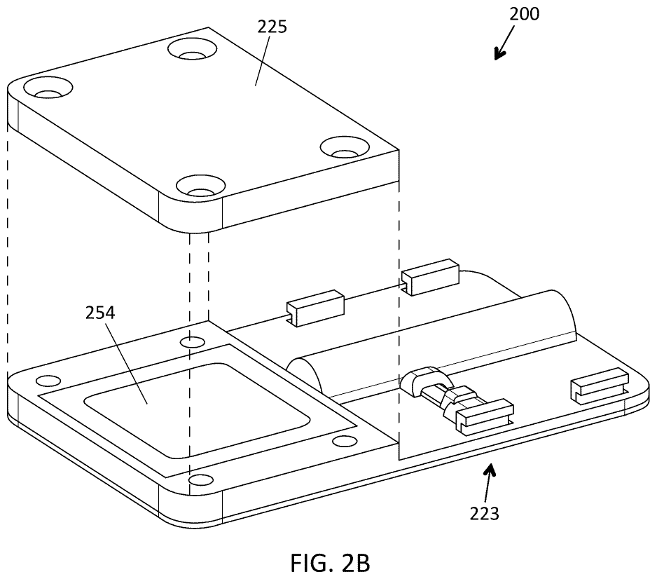

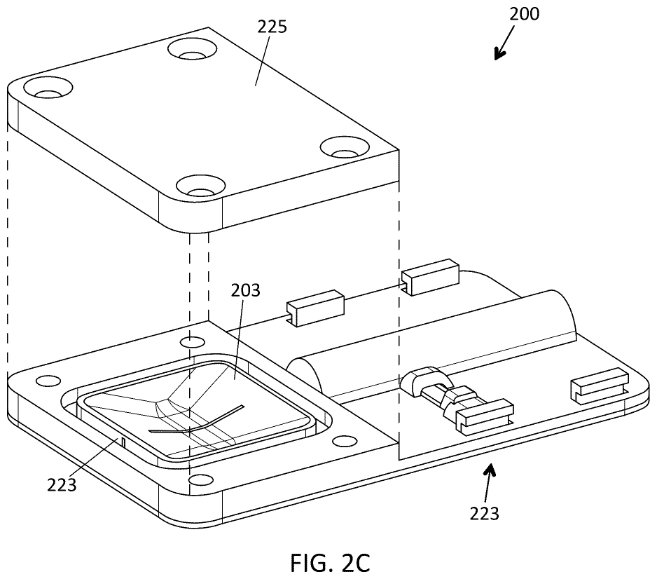

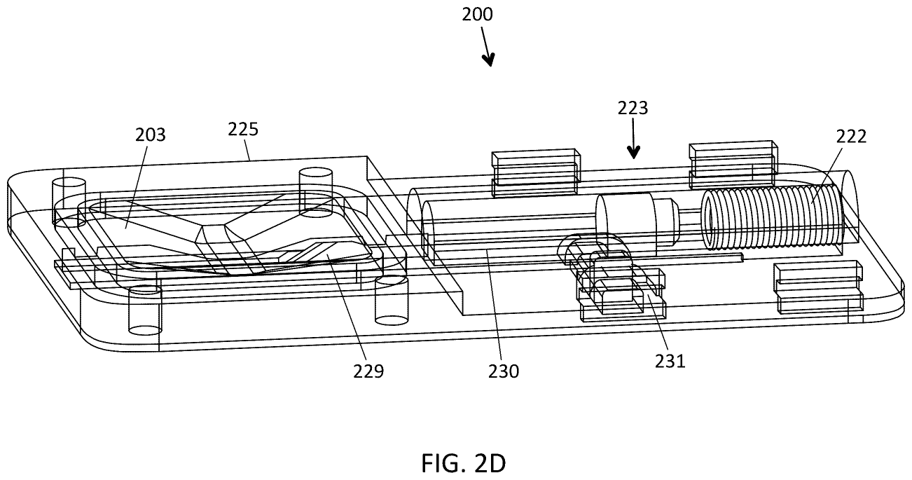

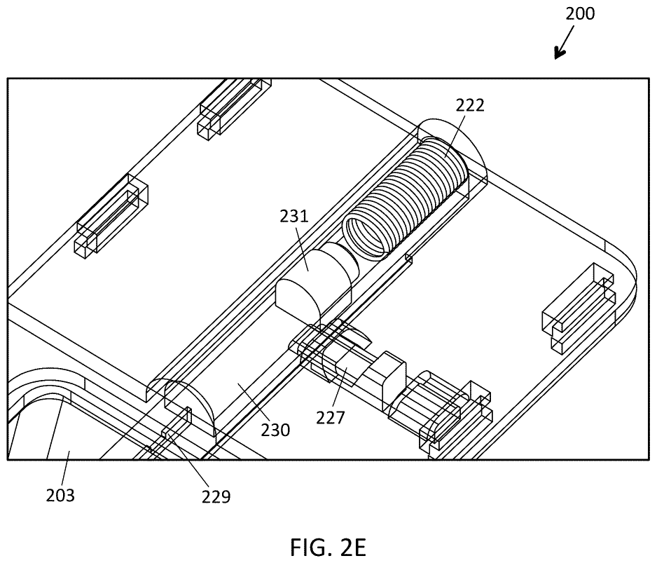

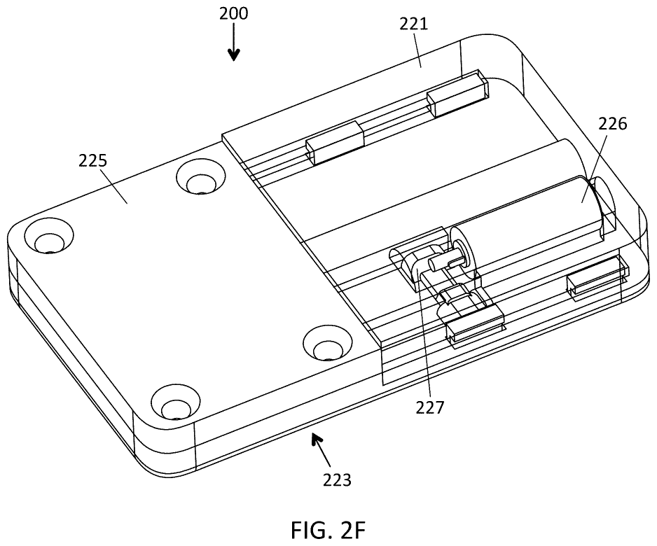

Referring to FIGS. 2A-2F, in some embodiments, the device 200 can be a two-part assembly including a reusable control unit 221 and a disposable cartridge 223. In some embodiments, the device 200 cannot be activated unless the control unit 221 and the cartridge 223 are connected together properly.

The disposable reservoir cartridge 223 can include a housing configured to connect to the control unit 221 and the drug reservoir 203. The cartridge 223 can further include a shuttle 230 with an attached blade 229, a compressed foam piece 254, a retainer 231, a spring 222, an adhesive membrane 205 (e.g., one specific to the drug formulation being used), and a cap 225 configured to cover the reservoir 203 and foam piece 254. In some embodiments, the drug reservoir 203 can be a flexible bag including the drug formulation. The drug reservoir 203 can be prefilled so that the dosage is accurate and safe.

The control unit 221 can be reusable and can include a motor 226, a cam 227, a power source (e.g., battery), a software control unit, and the interface/display. In some embodiments, the control unit 221 can be rechargeable. Further, in some embodiments, the control unit 221 can include a sensor, such as a compliance sensor. In some embodiments, the control unit 221 can be a mechanically automated device (i.e., can be non-digital and non-electronic).

When the control unit 221 and the cartridge 223 are assembled together, the drug reservoir 203 can be under constant pressure from the compressed foam piece 254. Before activation, the drug will not evacuate from the reservoir 203 because there is no egress route. Further, the compressed spring 222 can bias the retainer 231 to the forward position. When activated, the motor 226 can turn the cam 227, which moves the retainer 231 from the forward position to the back position and thus out of the path of the shuttle 230. The shuttle 230 with the attached blade 229 can then move along a track in the device as a result of force from the decompressing spring 222. As the shuttle 230 and blade 229 are moved forward, the reservoir 203 can be cut open by the blade 229. The reservoir 203, under constant pressure from the foam piece 254, can then eject the drug formulation through the opening in the reservoir 203. The shuttle 230 can have a hole therethrough that allows the fluid to travel therethrough. The ejected fluid can then be directed to the membrane 205.

In some embodiments, the device 200 can only deliver drug formulation once for each disposable cartridge 223. After the device 200 is disassembled, the disposable cartridge 223 can be thrown away and the control unit 221 recharged. The device 200 can deliver another bolus of drug formulation once a new disposable cartridge 223 is attached.

In some embodiments, the time of activation can be programmed by a smartphone paired to the device (e.g., via Bluetooth).

In some embodiments, the devices described herein can include additional chemical or mechanical permeation enhancers added to it. Further, in some embodiments, the device can include a needle for subcutaneous transdermal drug delivery.

In some embodiments, shuttle release mechanisms other than the described motor and cam can be used. For example, a shape-memory alloy (SMA) wire can be used to release the shuttle(s). The SMA wire can have a one-way memory effect that changes shape to a pre-defined shape upon heating. Thus, in use, a wire composed of SMA alloy can be connected to the retainer, a battery, and the housing of the disposable cartridge 223. When the device 200 is activated, the battery can run current through the SMA wire, allowing the wire to heat up through internal resistance. When heated past a certain temperature, the wire can deform to a secondary shape of shorter length, such as a coil or loop. The retainer 231 can thus be pulled along with the shortening wire out of the path of the shuttle 230, allowing for the shuttle 230 and blade 229 to pierce or cut the reservoir 203. In this embodiment, the compressed spring 222 that biases the retainer 231 to the forward position can be utilized or removed entirely. Moreover, the retainer 231 itself can be replaced with SMA wire if the wire acts as a retainer to the shuttle 230.

Additionally, in some embodiments, the reservoir can be cut through cutting mechanisms other than the described blade. For example, a needle can be attached to the moving shuttle to pierce the reservoir. The needle may also be used with another mechanism instead of the spring loaded shuttle. For example, the needle can remain stationary within the device and pierce the reservoir only when a protective agent is removed or when the reservoir is compressed with a certain amount of force to reach the needle. The cut or hole made by the needle can be small such that fluid is ejected with great force. This can be advantageous for wetting the entire membrane for delivery.

Additionally, in some embodiments, rather than using compressed foam to provide pressure to the reservoir, other methods of providing pressure can be used. For example, a leaf spring in either an elliptical or semi-elliptical shape can be used to provide constant downward force on the reservoir. As another example, a heat-expansive material that responds to heat generated within the device can be used to provide pressure on the reservoir.

In some embodiments, heat expansive foam can be used in conjunction with a moveable shuttle to improve the ejection of fluid.

In some embodiments, rather than including a single bolus, a drug delivery device can be configured to provide multiple boluses throughout the day, e.g., with the potential for an on-demand patient initiated dose.

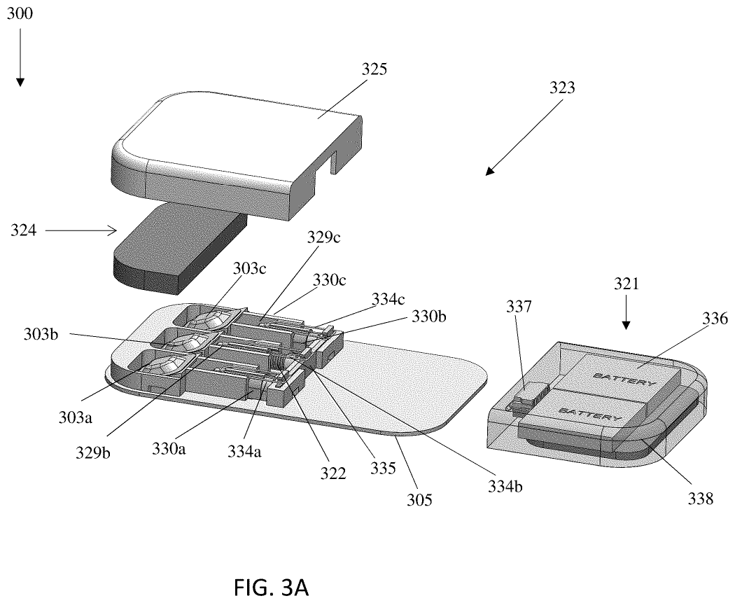

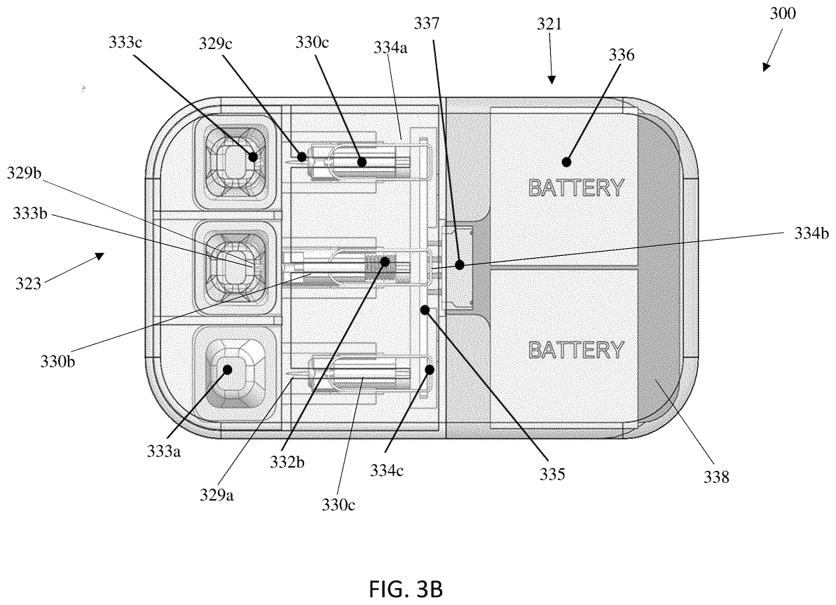

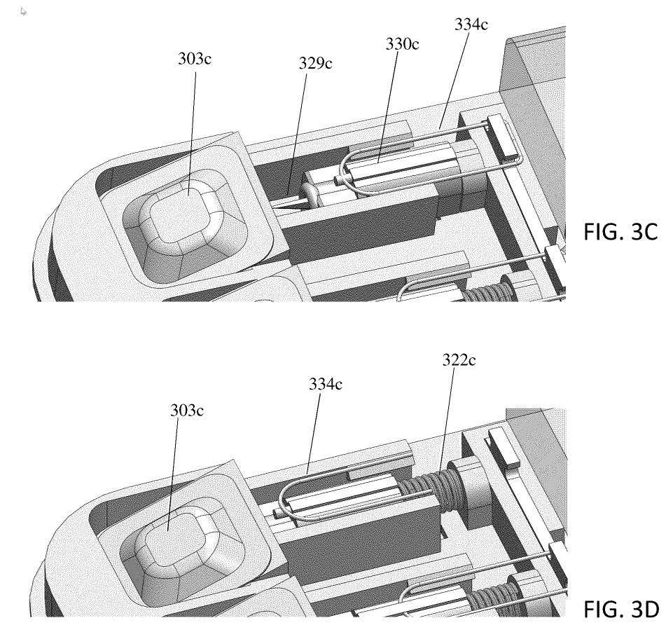

For example, FIGS. 3A-3D show a device 300 including a control unit 321 including a battery 336, a printed circuit board 338, and a connector 337 for connection to a multi-reservoir cartridge 323. The multi-reservoir cartridge 323 can include three fluid reservoirs 303a, b, c (e.g., sealed pouches or bags). Each reservoir 303a, b, c can include an outlet port 333a, b, c, e.g., under the reservoir 303a, b, c, to guide the drug formulation onto the membrane 305. The cartridge 323 can further include a sliding shuttle 330a, b, c and blade 329a, b, c aligned with each reservoir 303a, b, c. Further a corresponding spring 322a, b, c (322a, c are not shown for clarity) can be configured to decompress to activate each slide 330a, b, c. Foam 324 can be positioned between a cartridge cap 325 and the reservoirs 303a, b, c. Further, in some embodiments, the cartridge 323 include a release mechanism for holding the shuttles 330a, b, c in place till the time of actuation. For example, the release mechanism can be a fuse wire actuator, which can include a fuse wire 334a, b, c for each shuttle 330a, b, c.

Use of the device 300 is shown in FIGS. 3C-3D. Before use, the shuttle 330c is held in place with the fuse wire 334c (as shown in FIG. 3C). Upon combining the disposable cartridge 323 and the control unit 321, the control unit 321 is electrically joined to the disposable cartridge 323 such that current can be delivered to each fuse wire 334c independently upon actuation. Referring to FIG. 3D, electric current can break the fuse wire 334c, releasing the spring-loaded shuttle 330c (e.g., due to decompression of the spring 322c). As a result, the blade 329c can puncture the fluid reservoir 303c, allowing the drug therein to exit through the outlet port 333c. The drug formulation and solvent can then make contact with the transdermal drug delivery membrane 305. In some embodiments, the drug can pass through the membrane 305 into contact with the skin of the patient while the solvent evaporates from the membrane 305. Advantageously, the fuse wire release mechanism can allow separate activation of each shuttle, thereby allowing the individual reservoirs to be separately drained.

In some embodiments, the length, width, and thickness of the device 300 can be 40 mm-100 mm, 10 mm-70 mm, and 1 to 20 mm, respectively. For example, the length, width, and thickness of the design with fuse wire mechanism can be 73 mm, 42 mm, and 8.25 mm, respectively.

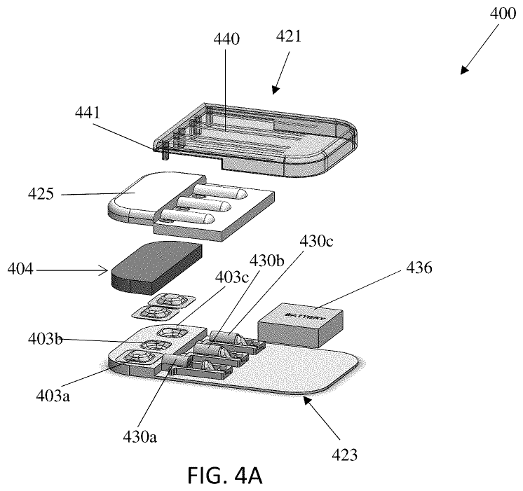

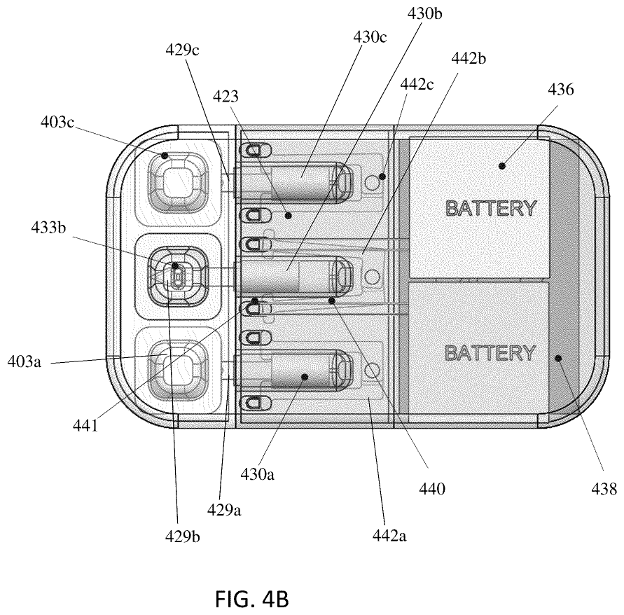

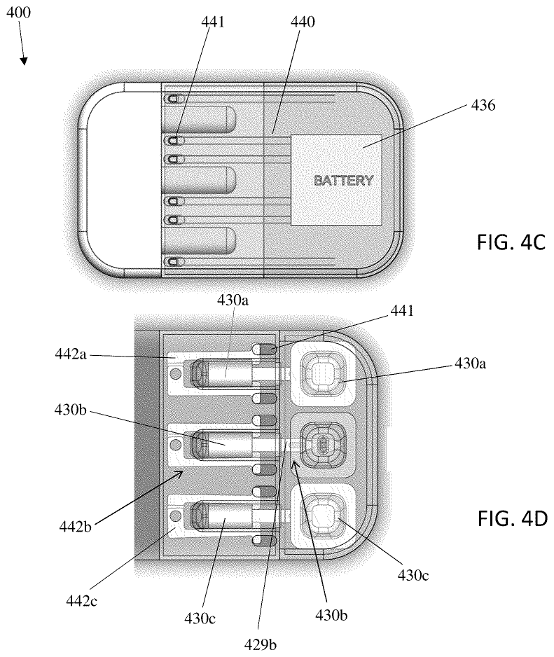

Another device 400 including multiple reservoirs is shown in FIGS. 4A-4D. The device 400 is similar to device 300 except that includes an SMA (shape memory alloy) wire actuator release mechanism. The device 400 thus includes a control unit 421 including a battery 436, a printed circuit board 438, and a connector for connection to a multi-reservoir cartridge 423. The multi-reservoir cartridge 423 can include three fluid reservoirs 403a, b, c (e.g., sealed bags). Each reservoir 403a, b, c can include an outlet port 433a, b, c, e.g., under the reservoir 403a, b, c to guide the drug formulation onto the membrane. The cartridge 423 can further include a spring-loaded sliding shuttle 430a, b, c and blade 429a, b, c aligned with each reservoir 403a, b, c. Foam 404 can be positioned between a cartridge cap 425 and the reservoirs 403a, b, c. In contrast to the fuse wire release mechanism of device 300, however, device 400 can include an SMA wire actuator release mechanism. The SMA wire actuator release mechanism includes SMA wires 440 in the control unit 421. The wires 440 can be wound around flexible posts 441. Further, each shuttle 430a, b, c can include a latch mechanism 442a, b, c therearound.

Use of the device 400 is shown in FIGS. 4C-4D. Before activation, the spring-loaded shuttles 430a, b, c are held in place by the latch mechanisms 442a, b, c. When electric current is applied from the battery 436 to the SMA wires 440, the SMA wires 440 contract, pulling on the flexible posts 441 and releasing the latches 442a, b, c. As a result, the shuttles 430a, b, c can move towards the reservoirs 403a, b, c, causing the blades 429a, b, c to puncture the reservoirs 403a, b, c and release the drug therein. In some embodiments, each shuttle 430a, b, c can be individually activated.

In some embodiments, the length, width, and thickness of the device 400 can be between 30 mm-100 mm, 10 mm-70 mm, and 1 mm to 15 mm, respectively. For example, the length, width, and thickness of the device 400 can be 70 mm, 42 mm, and 7 mm, respectively.

Devices 300 and 400 advantageously include no motor since actuation uses fuse wires or shape-memory ally (SMA) wires.

In some embodiments, where multiple reservoirs are used, the device can use a single track for a moveable shuttle that cuts multiple reservoirs at once or stops at multiple points in the track, cutting only a determined number of reservoirs upon each movement. Multiple shuttles moving along a single or multiple tracks may also be used when there is more than one reservoir.

Advantageously, by having multiple reservoirs, the device can deliver multiple boluses at once or deliver multiple boluses at separate points in time, depending on the need of the user. For example, one bolus can be delivered at five separate times in a day or three boluses can be delivered in the morning and two boluses delivered in the evening. In some embodiments, each reservoir can include a different volume of medication. Thus, when multiple reservoirs are present, the device can either be used to deliver more than one bolus of the same drug at different times (i.e., for prolonged concentration) or can be used to deliver distinct, but compatible drug formulations at the same time (e.g., for the combination of levodopa and carbidopa in the treatment of Parkinson's Disease).

Further, if multiple boluses and/or reservoirs are used, the relative size and shape of the device can change. The volume of each bolus can be smaller or the device itself can be larger if more than one bolus is to be used. For each separate time that one or multiple reservoirs is cut, a different needle or blade can be used so as to avoid contamination. One needle or blade may be used if all reservoirs are to be cut at the same time. The method of pressure application to the drug formulation reservoirs can either be universal or specific to each bag. For example, a larger piece of compressed foam that rests over the top of all bags can provide pressure to all of them or multiple pieces of compressed foam can rest above only one reservoir each.

In some embodiments, the devices described herein can have a rectangular shape with rounded corners. In other embodiments, the device can have an elliptical shape. In other embodiments, the device can be circular. Each of these designs can hold any combination of one or more reservoirs, one or more shuttles, and one or more tracks. Additionally, the fluid can be released from any position within the device (i.e., can be from the center or from other locations).

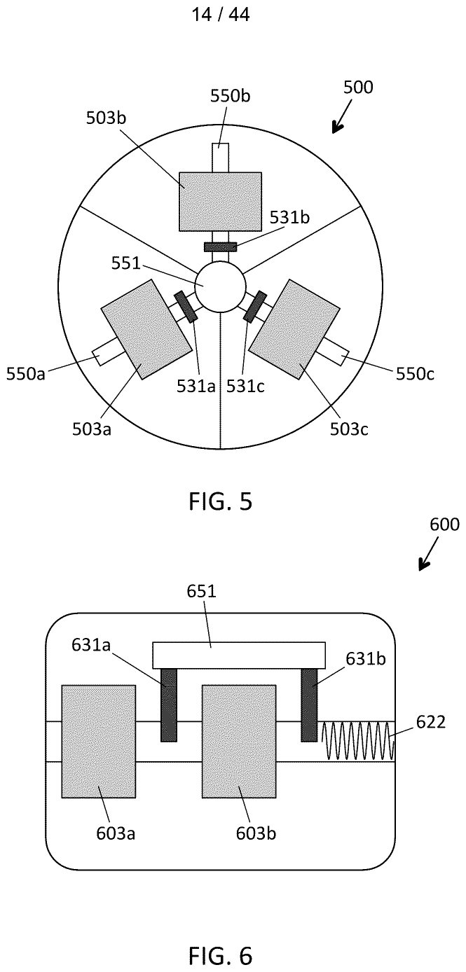

Exemplary devices 500, 600 with different shapes are shown in FIGS. 5 and 6. For example, FIG. 5 shows a device 500 with a circular shape. Three reservoirs 503a, b, c are positioned along tracks 550a, b, c along which the shuttles can travel. Retainers 531a, b, c keep the reservoirs 503a, b, c from opening prior to activation. A central release mechanism 551 can be configured to move the retainers 531a, b, c and release the shuttles along the tracks 5501a, b, c. FIG. 6 shows a rectangular device 600 with rounded corners. The device 600 includes two reservoirs 603a, b, a compressed spring 622 to bias the shuttles forward, and a shuttle release mechanism 651 configured to move retainers 631a, b out of the way of the shuttles when activated.

The devices described herein can be made of various materials and plastics. The majority of each device (e.g., the housing, the shuttle, the cam, the retainer, any other part) can be made from either the same material or from different materials. Each device may consist of or contain polypropylene, polyoxymethylene, low density polyethylene, high density polyethylene, ethyl vinyl acetate, polyester, polyethylene terephthalate, polyvinyl chloride, polyvinylidene chloride, polystyrene, polyamides, acrylonitrile, polycarbonate, polyurethane, polyimide, silicone or any other material.

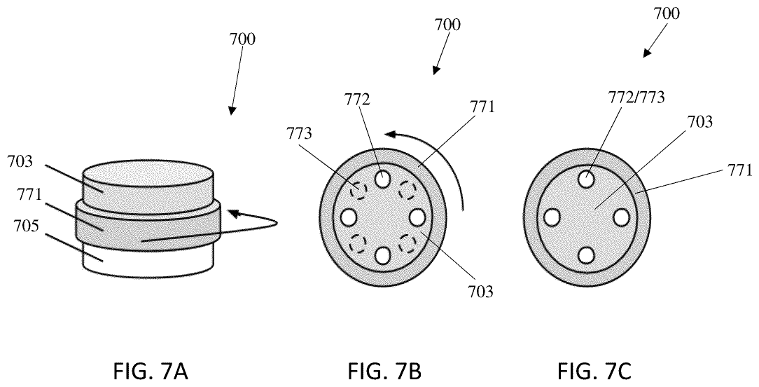

Another exemplary transdermal delivery device is shown in FIGS. 7A-7C. The device 700 includes a transdermal adhesive membrane 705, a drug reservoir 703, and a barrier 771 therebeween. The bottom of the reservoir 703 includes a pattern of holes 772. Further, the barrier 771 includes the same pattern of holes 773, but the pattern is initially rotationally offset such that the drug cannot flow from the reservoir 703 through the barrier 771. Once activated, however, the barrier 771 and reservoir 703 can rotate relative to one another so as to align the holes 772/773. As a result, drug can flow through the holes 772/773 to the membrane 705 (e.g., a transdermal adhesive membrane) and thus to the patient. The barrier 771 can fit tight and/or be sealed to the reservoir 703, thereby preventing or minimizing leaks.

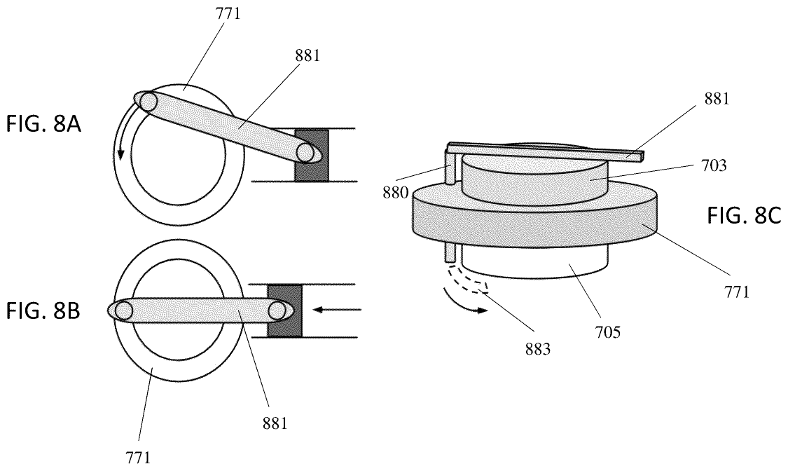

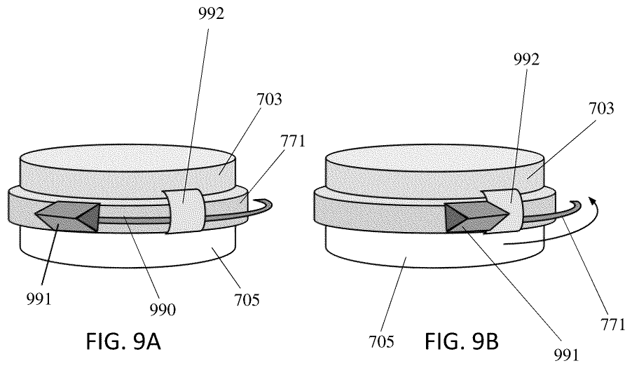

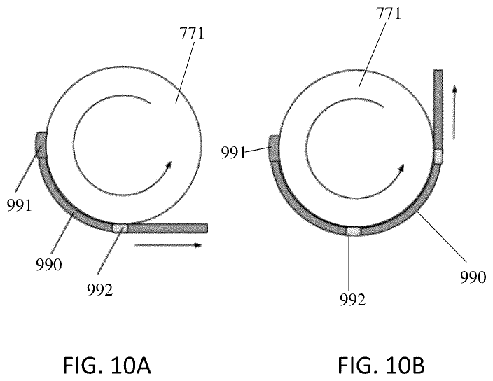

The reservoir 703 and barrier 771 can be rotated relative to one another by various mechanisms. In one embodiment, referring to FIGS. 8A-8C, a crank mechanism can be used. A vertical rod 880 can thus be fixed on the edge of the barrier 771. The vertical rod 880 can be connected to a horizontal rod 881, which can be connected to a piston. When a pushing force is applied to the piston 882, the barrier-connected end of the horizontal rod 881 can move in a circular orientation, causing the barrier 771 to turn. The bottom of the vertical rod 880 can sit in a small, circular groove 883, which can limit the rotation of the barrier 771 so that it cannot turn beyond the desired angle. In another embodiment, referring to FIGS. 9A-9B, a pulley system can be used. A wire 990 can wrap around part of the barrier 771 (the barrier can have a slight groove that prevents the wire 990 from coming off of the barrier). A small loop 992 can attach to the barrier 771 over the wire 990. Further, the wire 990 can have a large end piece 991 configured so as to not fit through the loop 992. When a pulling force is applied to the wire 990, the barrier 771 can turn until the end piece 991 makes contact with the loop 992. In some embodiments, the pulley system can be used in tension. In some embodiments, the pulley can use high resistance wire to burn the pulley wire and cause it to snap, thereby allowing the barrier 771 to rotate. Referring to FIGS. 10A-10B, in some embodiments, the wire 990 can be pulled straight back from the loop 992 (FIG. 10A) or the wire 990 can wrap around the barrier 771 before being pulled (FIG. 10B).

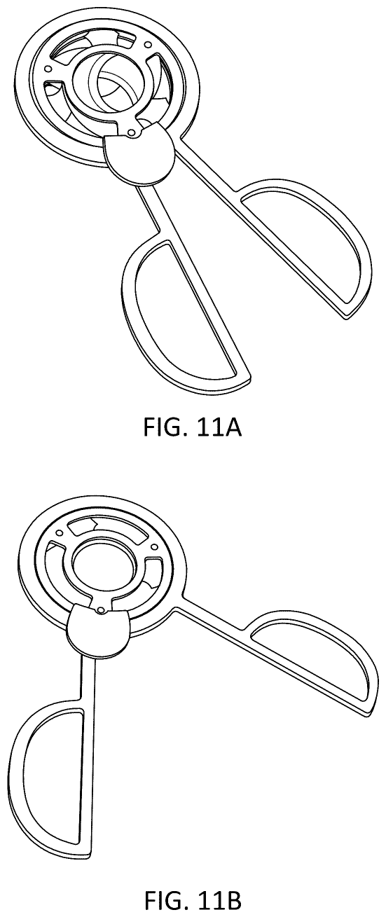

Referring to FIGS. 11A-11B, in some embodiments, the barrier and reservoir can have a windmill-type design.

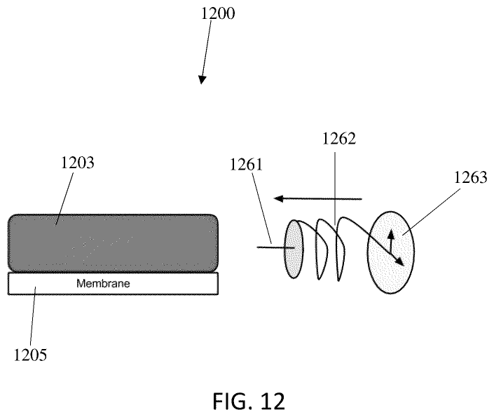

Another exemplary transdermal delivery device is shown in FIG. 12. The device 1200 includes the drug reservoir 1203 and membrane 1205, a needle 1261, spring 1262, and timer 1263. At the appropriate time (as set by the timer 1263), the needle 1261 attached to the spring 1262 can puncture the reservoir 1203, allowing the drug to flow through the membrane 1205. In some embodiments, a heat-expansive material can be used rather than a spring 1262.

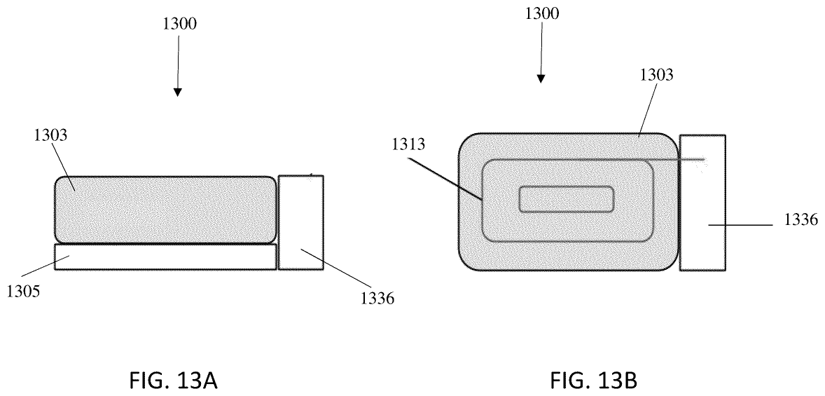

Another exemplary transdermal delivery device is shown in FIGS. 13A and 13B. The device 1300 includes a meltable drug reservoir 1303 and resistance wire 1313 patterned along the bottom of the reservoir 1303. When electricity is applied to the wire 1313 from the battery 1336, the wire 1313 can heat up and cause the reservoir 1303 to puncture or melt. Once punctured, the drug can flow from the reservoir 1303 through the membrane 1305. In some embodiments, the wire 1313 can be in contact with the membrane 1305. In other embodiments, the wire 1313 can be spaced way from the membrane 1305 (for example, either in a different location on the drug reservoir or separated by a spacer with holes).

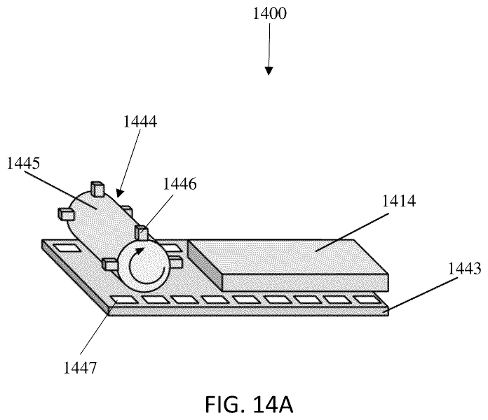

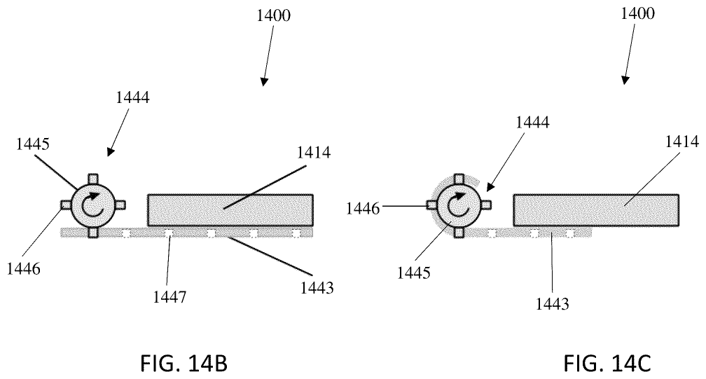

Another exemplary transdermal delivery device is shown in FIGS. 14A-14C. The delivery device 1400 includes a gel patch 1414 where the drug and adhesive are combined in a gel-like substance. The gel patch 1414 is separated from the skin by a thin liner 1443. The liner 1443 can be made of a flexible plastic and can be slightly lubricated on both sides in order to allow it to slide easily. One end of the liner 1443 can include slits 1447 therein. Further, an applicator 1444 for the device can include a cylinder 1445 with hooks 1446. The hooks 1446 can catch on the liner 1443 through the slits 1447 such that the liner 1443 can be removed as the cylinder 1445 is turned. Once the entire liner 1443 is spun around the cylinder 1445, the gel patch 1414 can lie directly on the skin. In some embodiments, the applicator 1444 can be used to remove a barrier between the drug reservoir and the membrane. In some embodiments, the liner 1443 can be twisted or folded for removal.

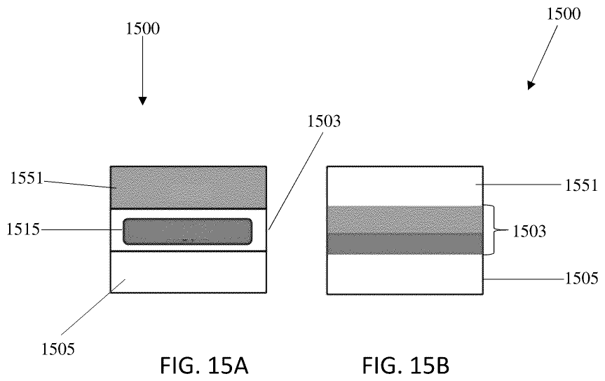

Another exemplary transdermal delivery device is shown in FIGS. 15A-15B. The device 1500 includes a reservoir 1503 having a pod 1515 holding the drug therein and a solvent reservoir 1551. The pod 1515 can be made of a material, such as polyvinyl alcohol, that can dissolve upon interaction with a solvent, such as water. The device 1500 can work by releasing the solvent from the solvent reservoir 1551 into the drug reservoir 1503, resulting in the dissolution of the pod 1515 and release of the drug to the membrane 1505 and thus to the skin. In some embodiments, the pod 1515 can be a gel capsule and the solvent an acidic solution.

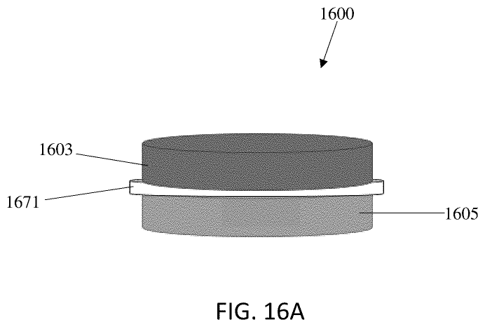

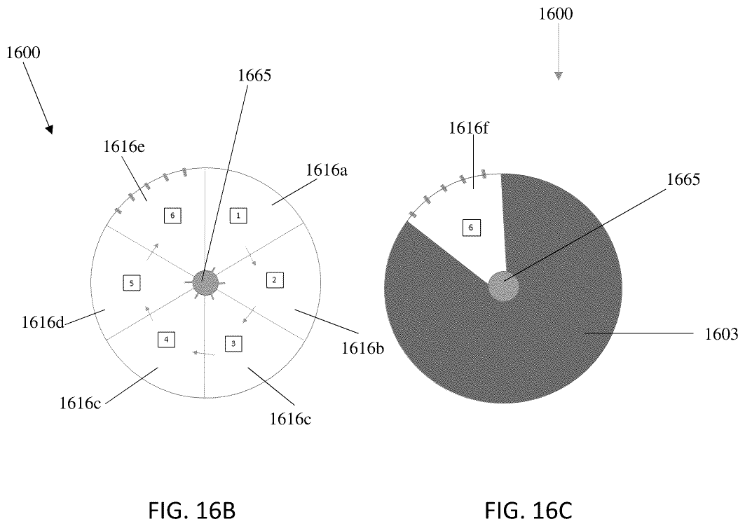

Another exemplary transdermal delivery device is shown in FIGS. 16A-16C. In this embodiment, the device 1600 includes a drug reservoir 1603, a barrier 1671, and a membrane 1605. The barrier 1671 can be configured as a ‘rotating fan’ with a plurality of panes 1616a, b, c, d, e. The barrier 1671 can be made of as many or as few panes as desired. Each pane can be secured to a central, permanent, small cylinder 1665 at varying heights. One of the panes 1616 can be secured permanently in place relative to the cylinder 1665. In use, the control unit can be placed on top of the cartridge (including the reservoir 1603 and barrier 1671) and can rotate the central cylinder 1665 to move the panes until motion is complete and the panes 1616a-f are all aligned under the permanent pane 1616.

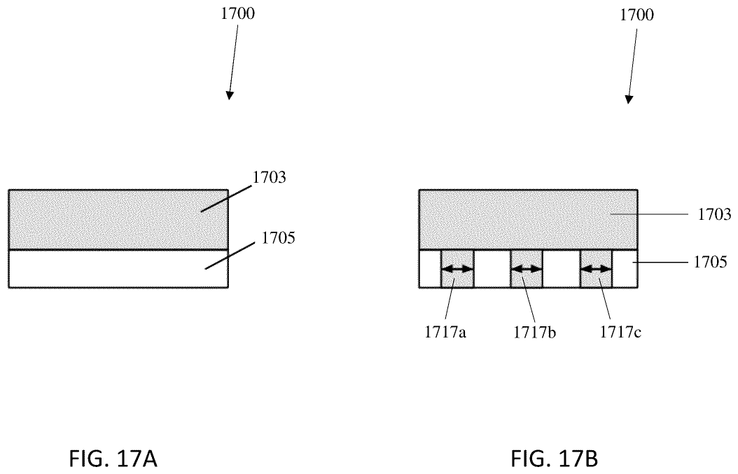

Another exemplary transdermal drug delivery device is shown in FIGS. 17A-17B. The device 1700 includes an open reservoir 1703 in which the drug therein sits directly on the membrane 1705. The membrane 1705, however, does not initially allow flow therethrough. Upon activation, the pores 1717a, b, c in the membrane 1705 can expand, allowing the drug to travel therethrough to the skin. The pores 1717a-c can be designed to expand the same amount each time activated, thereby providing consistent dosage. In some embodiments, the membrane 1705 can be made of a fully-hydrated lipid bilayer, and a peptide, such as melittin, can be used to induce the pores 1717a-c to widen. In some embodiments, the membrane 1705 can be a cell membrane, and channel proteins can be used to form temporary pores 1717a-c.

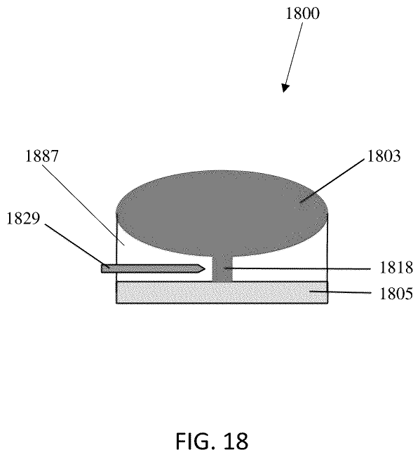

Another exemplary transdermal drug delivery device is shown in FIG. 18. The device includes a reservoir 1803. The reservoir 1803 can have a blow fill seal 1818 that leads to the membrane 1805. The device 1800 can further include a blade 1829 embedded in the housing 1887 (e.g., plastic housing). Upon activation, the blade 1829 can be pushed forward (e.g., by a cam of the control unit) to cut through the seal 1818 and release the drug onto the membrane 1805. In some embodiments, the control unit can include a leaf spring about the size of the reservoir 1803. The leaf spring can exert a pre-loaded force on the reservoir to ensure emptying of the reservoir.

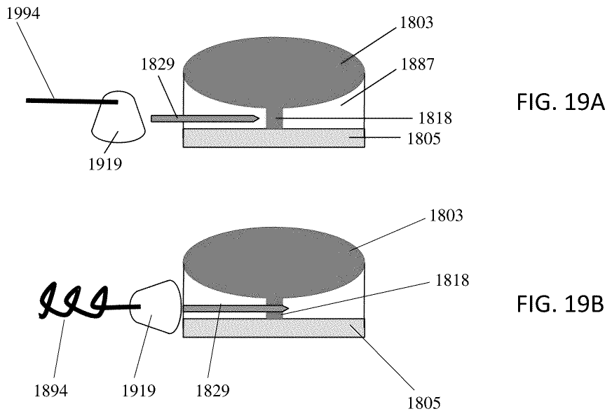

In some embodiments, as shown in FIGS. 19A-19B, the blade 1829 can be activated by a cam 1919. For example, a straight segment of SMA wire 1994 can be looped through the small end of the cam 1919. Upon activation, the wire 1994 can be heated so as to transition from the straight configuration (see FIG. 19A) to a coiled configuration (see FIG. 19B). As the wire 1994 coils, it can rotate the cam 1919, which can push the blade 1929 forward to cut the seal 1818. In some embodiments, a board switch can actuate the cam 1919 rather than the wire.

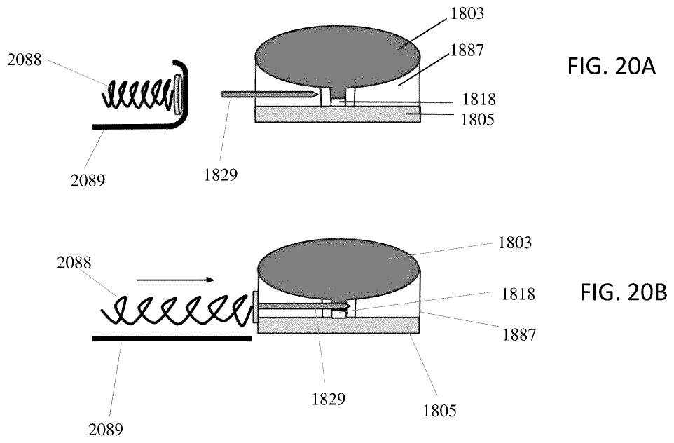

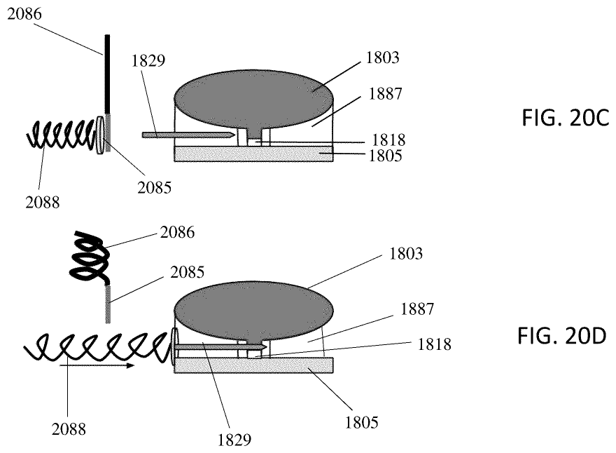

In some embodiments, as shown in FIGS. 20A-20D, the blade 1829 can be activated by a compressed spring 2088 and SMA wire 2089. For example, and as shown in FIGS. 20A-20B, the spring 2088 can be held in place by a piece of flat SMA wire 2089 (both of which can be components of the control unit). Upon activation, the SMA wire 2089 can be heated and straighten to release the spring 2088. The spring 2088 can push the blade 1829 into the seal 1818. As another example, and as shown in FIGS. 20C-20D, the spring 2088 can be held in place by a thin block 2085 attached to a straight SMA wire 1086 (which can be part of the control unit along with the spring 2088). Upon activation, the SMA wire 2086 can be heated, transitioning it to a coiled configuration and moving the block 2085 out of the way of the spring 1088, thereby releasing the spring 2088 and pushing the blade 1829 forward to cut through the seal 1818.

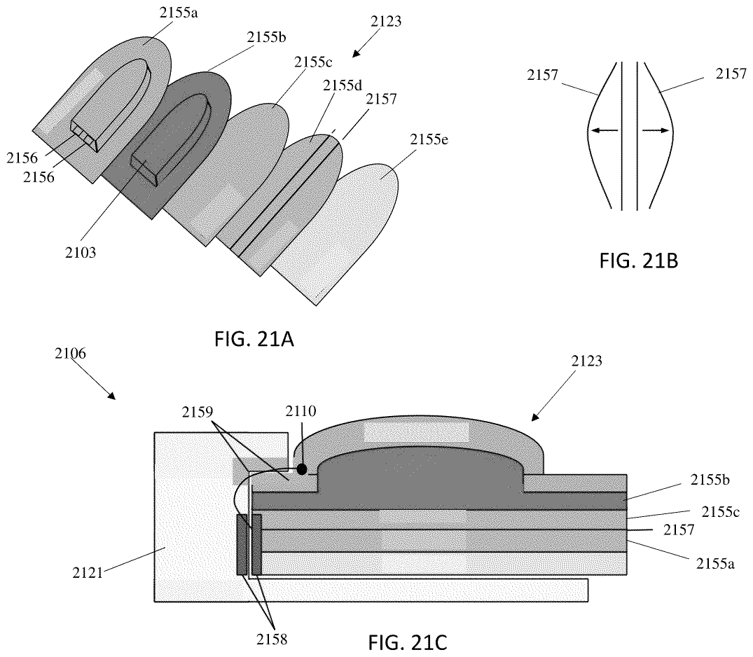

Another embodiment of a transdermal delivery system is shown in FIGS. 21A-21C. The device 2100 can include a control unit 2121 and a cartridge 2123. The cartridge 2123 can be made of a plurality of layers 2155a-e. The top layer 2155a can be a casing, such as a plastic casing, and can include metal contacts 2156 or plates configured to connect to metallic plates 2159 of the control unit 2121. The second layer 2155b can include an open drug reservoir 2103. The second layer 2155b can be made, for example, of a plastic such as formed polyethylene terephthalate (PETG). The third and fourth layers 2155c, d can be made of film or foil and can have two parallel SMA wires 2157 therebetween. The wires 2157 can be configured to connect to the metal contacts 2156 on the first layer 2155a (e.g., with solder 2110). Upon application of electricity from the control unit 2121, the wires 2157 can heat up, changing the shape of the wires 2157 (as shown in FIG. 21B) and causing the layers 2155 c, d to tear and therefore allow drug to flow from the reservoir 2103 to the bottom layer 2155c (e.g., the adhesive membrane). In some embodiments, the control unit 2121 and cartridge 2123 can be connected together with magnets 2158.

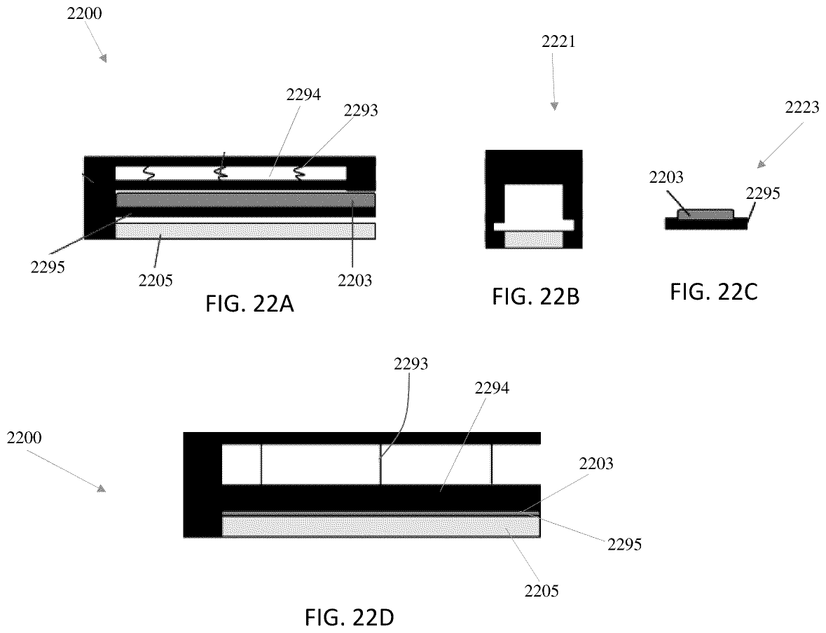

Another embodiment of a transdermal drug delivery system is shown in FIGS. 22A-22D. The device 2200 can include a cartridge 2223 and control unit 2221. The cartridge 2223 can include a reservoir 2203 (e.g., in the form of a blister) adhered to a porous separator 2295. The cartridge 2223 can be configured to fit within the control unit 2221 and can be held in place by grooves on the side of the product. The control unit 2221 can include a plurality of SMA coils 2293 that, upon activation (e.g. via current from a battery), the coils 2293 can extend from a coiled or bent configuration to a straighter configuration, pushing into the reservoir 2203 (e.g., through a flat plate 2294). The pressure of the flat plate 2294 and the porous separator 2295 can cause the reservoir 2203 to burst or pop due to pressure. The drug can then be released through the separator 2295 and the membrane 2205.

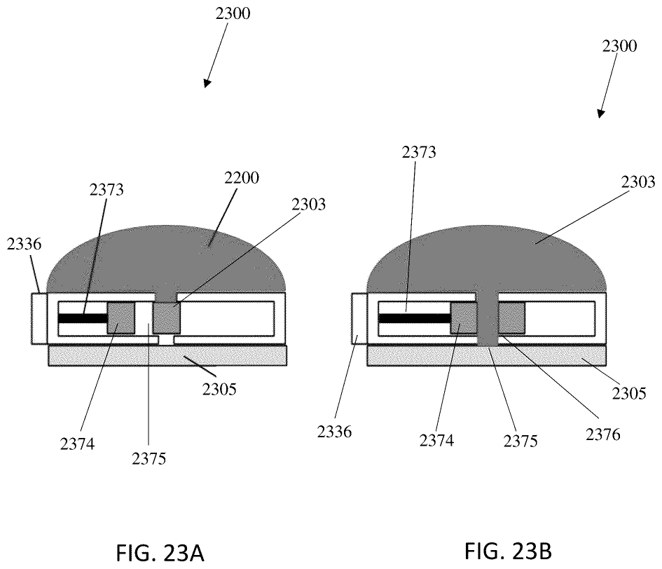

Another exemplary embodiment of a transdermal drug delivery device is shown in FIGS. 23A-23B. The device 2300 includes a reservoir 2303, a resistance wire 2373, and a block 2374 with a hole 2375 therethrough. Before activation (FIG. 23A), the block 2374 is positioned so as to block drug from flowing through the reservoir opening 2376. Upon activation (FIG. 23B), the battery 2336 can heat the resistance wire 2373, causing the wire 2373 to expand and push the block 2374 such that the hole 2375 and the outlet 2376 align, thereby allowing the drug to flow therethrough and into the membrane 2305.

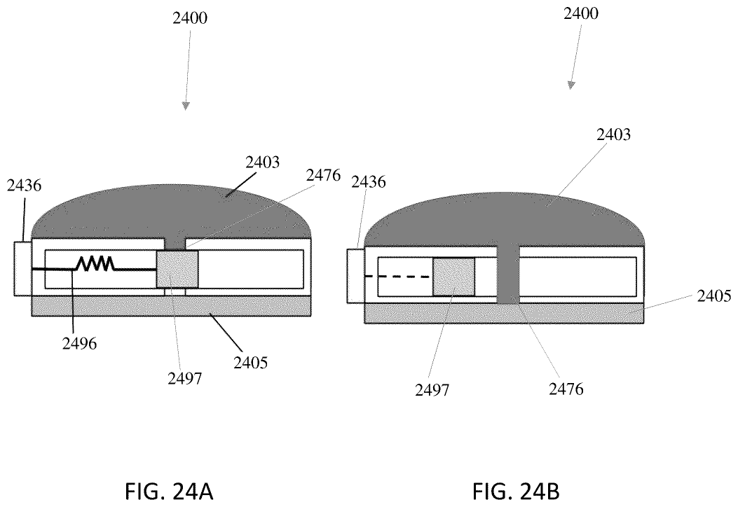

Another exemplary embodiment of a transdermal drug delivery device is shown in FIGS. 24A-24B. The device 2400 includes a reservoir 2403 and a resistance wire 2496 configured to apply tension to a block 2497, which blocks the reservoir outlet 2476 prior to activation (FIG. 24A). When current is applied from the battery, the resistance wire 2496 can burn, thus releasing the block 2497 from tension and allowing it to move away from the outlet 2476 (FIG. 24B). Drug can then flow from the reservoir 2403, through the outlet 2476, and onto the membrane 2405.

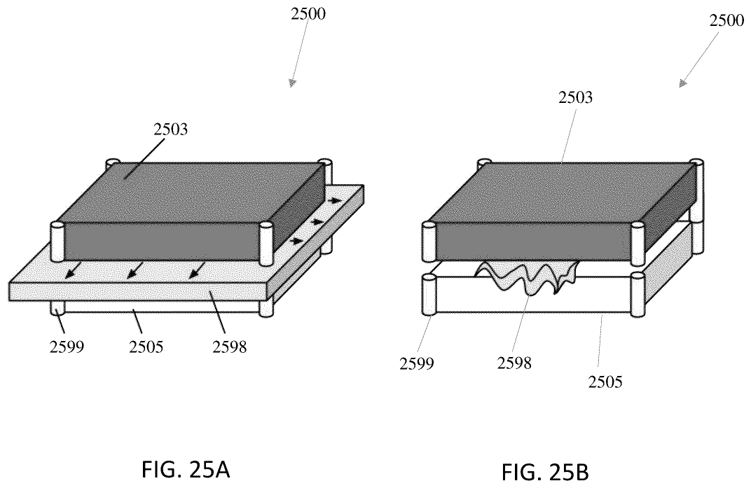

Another exemplary embodiment of a transdermal drug delivery device is shown in FIGS. 25A-25B. The device 2500 includes an open reservoir 2503, a membrane 2503, and a thin barrier 2598. The barrier 2598 can be made of a meltable material and can be stretched tightly between the corners of the device 2500. A plurality of micro-heaters 2599 can be positioned around the device 2500 in contact with the barrier 2598. Upon activation (from FIG. 25A to FIG. 25B), the micro-heaters 2599 can melt the barrier 2598, causing it to lose its tension and collapse. The collapsed barrier 2598 can then allow the drug to flow from the reservoir 2503 onto the membrane 2505. The number of micro-heaters 2599 can vary (e.g., 1, 2, 3, 4, or more than 4 can be used).

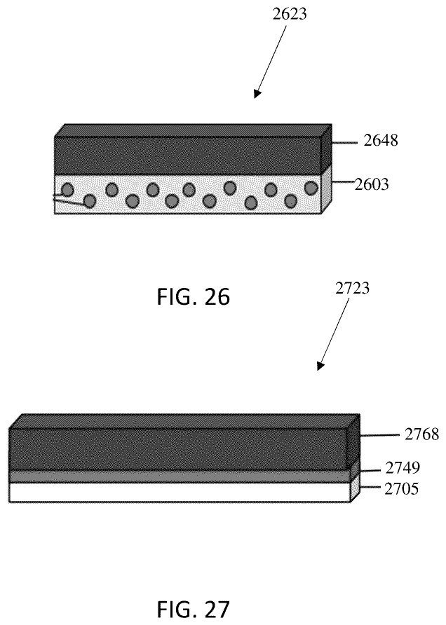

Another exemplary cartridge for a transdermal delivery device is shown in FIG. 26. The cartridge 2623 includes a solvent reservoir 2648 and a drug reservoir 2603. The solvent reservoir 2648 can include water or other solvent while the drug reservoir can include a hydrated hydrogel, alcoholic gel, or hydro-alcoholic gel incorporating the drug therein. Upon activation, the breaking element can cut open the reservoir and the solvent reservoir 2648 can release the solvent into the drug reservoir 2648, which can allow the drug to release to the patient's skin (e.g., through an adhesive membrane).

Another exemplary cartridge for a transdermal delivery device is shown in FIG. 27. The cartridge 2723 can include a solvent reservoir 2768, a membrane 2705, and a layer 2749 of dehydrated drug therebetween. Upon activation, the solvent can be released from the reservoir 2748 to rehydrate the drug and allow it to pass through the membrane 2705.

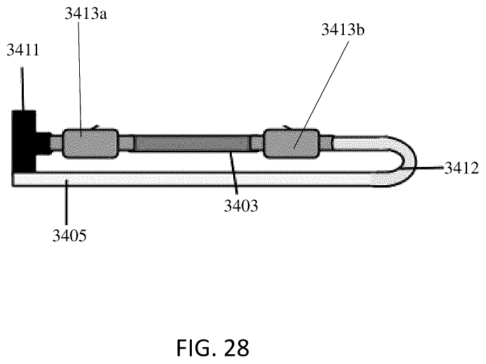

Another exemplary embodiment of a transdermal drug delivery device is shown in FIG. 28. The device 3400 includes a drug reservoir 3403 and a membrane 3405. Additionally, a microblower 3411 is fluidically connected to the reservoir 3403 through tubing 3412. Check valves 3413a, b are positioned within the tubing 3412 on either side of the reservoir 3403. Upon activation, the microblower 3411 can provide the necessary force to push the drug from the reservoir 3403 through the tubing 3412 and onto the membrane 3405. The microblower 3411 can be made, for example, of small piezoelectric elements. The check valves 3413a, b can prevent the premature loss of drug from the reservoir 3413a, b and can provide for fluid travel in only a single direction (i.e., from the reservoir 3403 to the membrane 3405). In some embodiments, the device 3400 can be made of a separate control unit and cartridge. The control unit, for example, can include the microblower 3411 while the cartridge can include the check valves 3413a, b. The device 3400 advantageously uses air as a safe and reliable plunger to convert electrical energy to mechanical energy.

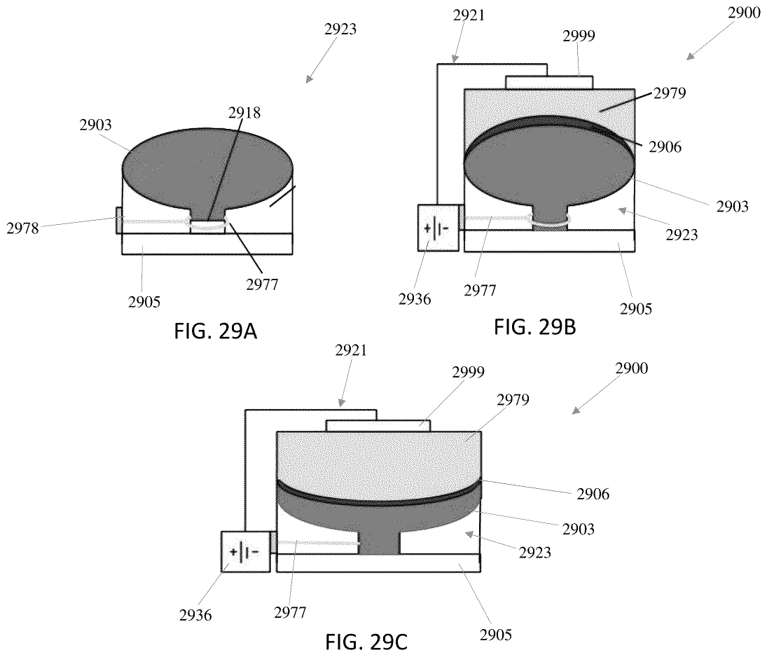

Another exemplary transdermal drug delivery device is shown in FIGS. 29A-29C. The device 2900 includes a cartridge 2923 and a control unit 2921. The cartridge 2923 includes a drug reservoir 2903 that is closed with a blow-fill seal 2918. Around the seal is a high-resistance wire 2977, which is connected to a metal contact 2978. The control unit 2921 includes a battery 2936, a micro-heater 2999, and a heat-expansive material 2979 that is sealed with a stretch membrane 2906. Upon activation, battery 2936 can provide current to the wire 2977 to melt the blow-fill seal 2918. Simultaneously, the battery 2936 can activate the micro-heater 2999, which can expand the heat-expansive material 2979, causing the stretch membrane 2906 to stretch, pushing against the reservoir 2903 and forcing the drug out of the reservoir 2903 and onto the membrane 2905 (transition from FIG. 29B to FIG. 29C). In some embodiments, no heat-expansive material is used, and the drug evacuates the reservoir 2903 through capillary action.

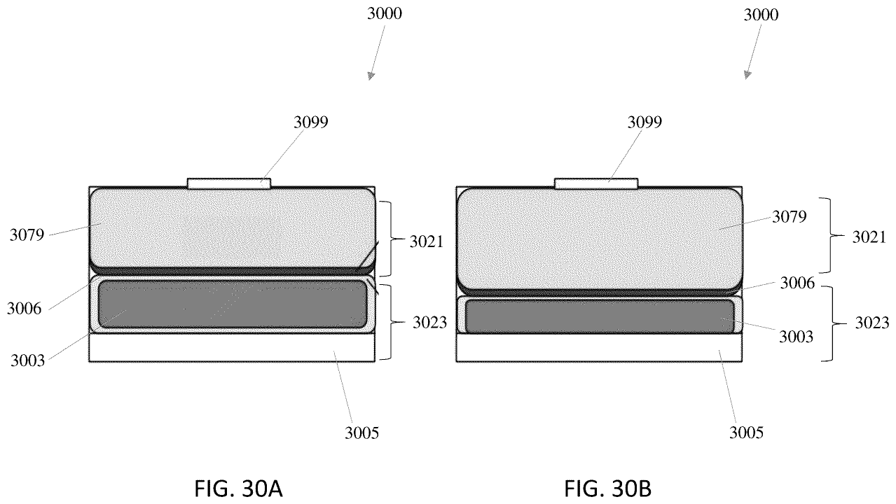

Another exemplary transdermal drug delivery device is shown in FIGS. 30A-30B. The device 3000 includes a control unit 3021 and a cartridge 3023. The cartridge 3023 includes a drug reservoir 3003 that is enclosed by a thin “burst” casing. The control unit 3021 includes a micro-heater 3099, a heat-expansive material 3079, and a burst membrane 3006. Upon activation, the micro-heater 3099 heats up, causing the heat-expansive material 3079 to expand and stretch the stretch membrane 3006. As a result of the pressure from the membrane 3006, the burst casing of the reservoir 3023 can pop, allowing drug to flow to the membrane 3005.

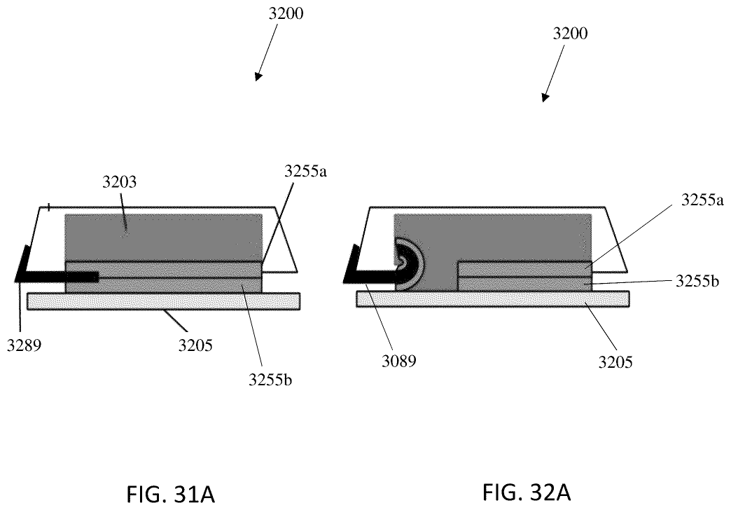

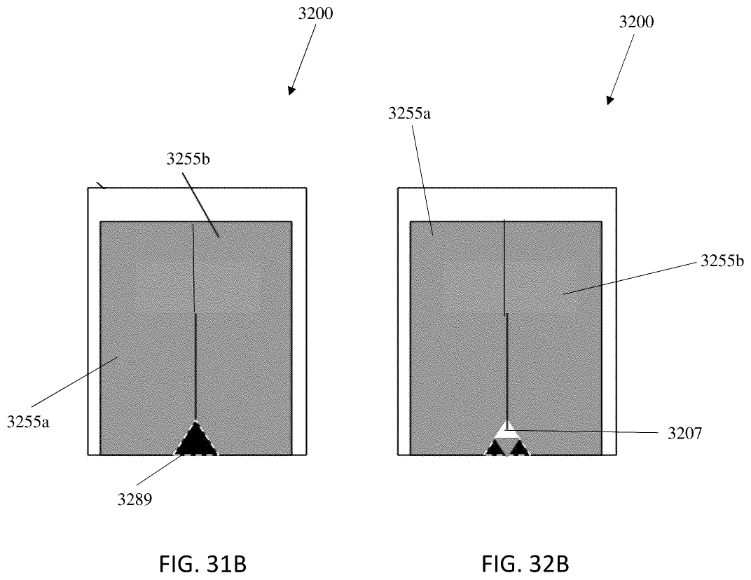

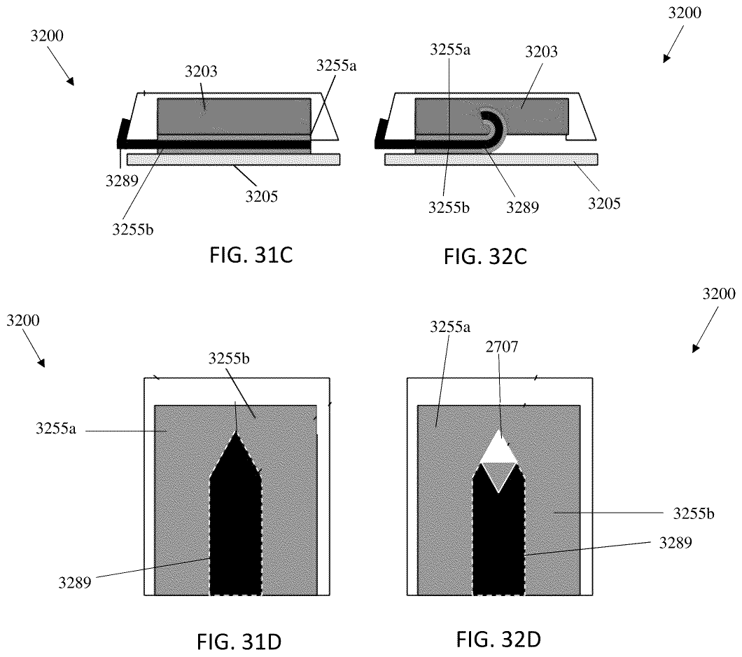

Another exemplary transdermal drug delivery device is shown in FIGS. 31A-3D (inactivated) and FIGS. 32A-D (activated). The drug delivery device 3200 includes an open drug reservoir 3203 positioned over two layers 3255a, b of foil. A flat SMA wire 3259 is positioned horizontally between (e.g., parallel with) the layers 3255a, b. Upon activation, current can be applied to the SMA wire 3259, causing it to heat and curl (see the transition from FIG. 31A-32A). As the wire 3259 curls, the tip (e.g., pointed tip) of the wire can puncture the layers of foil 3255a, b (see the transition from FIGS. 31B-32B). As a result, a large slit 3207 can form in the layers 3055a, b. The drug can then flow from the reservoir 3203 onto the membrane 3205. As shown in FIGS. 31C-D and 32C-D, in some embodiments, the wire 3259 can be thicker and/or extend further between the layers 3255a, b.

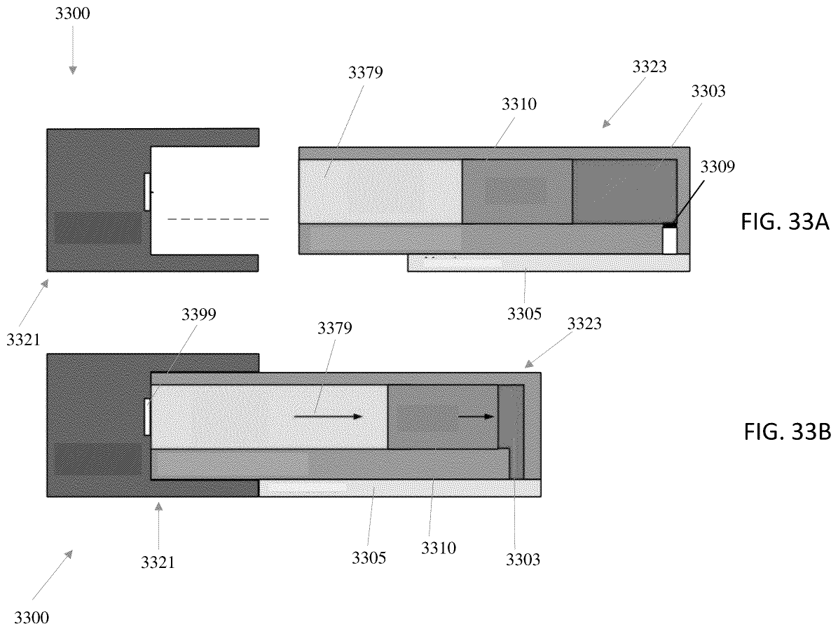

Another exemplary embodiment of a transdermal drug delivery device is shown in FIGS. 33A-33B. The device 3300 includes a control unit 3321 and a cartridge 3323. The cartridge 3300 includes a drug reservoir 3303 that is connected to membrane 3305 through a tube 3308. A thin film 3309 sits inside the tube 3308 at the junction with the reservoir 3303. The cartridge 3300 further includes a plunger 3310 (e.g., a plunger made of an insulated material). The plunger 3310 can include a seal therearound at the junction with the drug reservoir to prevent the reservoir 3303 from leaking. The control unit 3321 can include a micro-heater 3399. Further, either the control unit 3321 or the cartridge 3332 can include a heat expansive material 3379. Upon activation, the micro-heater 3399 can heat the heat-expansive material 3379, causing it to expand and push the plunger 3310 further into the reservoir 3303. As the plunger 3310 moves into the plunger it can break through the thin film 3309 and allow the drug to flow from the reservoir 3303 onto the membrane 3305.

Any of the transdermal delivery devices described herein can include separate cartridges and control units.

The transdermal delivery devices described herein can be small. For example, the devices described herein can have a height (i.e., distance from the skin) of less than or equal to 7 mm.

The reservoirs described herein can be configured to hold, for example, 1 μl or more, 2 μl or more, 3 μl or more, 4 μl or more, or 5 μl or more of fluid. By holding only a limited amount of fluid, the cartridges and/or devices described herein can reduce the potential for overdose.

Any of the transdermal drug delivery devices described herein can be configured to be activated at a set time (e.g., just prior to wake-up). Advantageously, using the pre-set timed delivery of drug formulation allows the patient to have optimized treatment with increased adherence and compliance since the drug dosing is automated and programmable. After a user sets the time of delivery and puts on the device, the user advantageously need not worry about taking pills or injecting solution at inopportune times. This enables, for example, the user to get a full night of sleep without waking up to take medication.

Advantageously, the devices described herein allows the delivery of medication without needles, microneedles, thermal ablation, iontophoresis or any other stratum corneum disrupting technique and allow the user to be medicated without pain or discomfort.

Advantageously, the devices described herein can be used with disposable cartridges, which can be filled with different medications and/or doses depending upon need.

Apparatuses, systems and methods disclosed herein can have improvements over current state-of-the-art such as smaller volume, lower vapor losses, simpler mechanism and variable bolus volume. For example, fluid-filled bags as reservoirs can have potentially low vapor losses because of no sliding fluid interfaces in some embodiments.

Although the medical devices described herein have been described for use in smoking cessation, it is to be understood that they could be used for other types of treatment and medications as well, such as treatment of allergies, ADHD, and Parkinsons.

It should be understood that any features described with respect to one embodiment herein can be combined or substituted for any feature described with respect to another embodiment. For example, where a breaking elements is described as any of a blade, a needle, an SMA wire, or a solvent dissolving reservoir, the piercing elements can be substituted for any other piercing element.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system for transdermal delivery of a drug formulation comprising:
a transdermal membrane;
a drug cartridge including:
a sealed reservoir configured to hold a drug formulation therein;
a breaking element configured to break the sealed reservoir upon actuation;
an actuator disposed on a first side of the sealed reservoir configured to activate the breaking element to break the sealed reservoir; and
a pressurizing element configured to place pressure on a second side the sealed reservoir opposite the first side such that, upon activation of the breaking element, drug formulation from the sealed reservoir is forced out of the sealed reservoir and onto the transdermal membrane; and
a control unit having a battery and a printed circuit board for actuation of the actuator.

2. The system of claim 1, wherein the breaking element is configured to pierce the sealed reservoir when heated.

3. The system of claim 1, wherein the breaking element includes a shape-memory alloy wire configured to lengthen when heated.

4. The system of claim 1 wherein the breaking element is a wire configured to extend and pierce the sealed reservoir as an electrical current is passed through the wire.

5. The system of claim 1, wherein the pressurizing element is configured to place a constant pressure on the sealed reservoir.

6. The system of claim 1, wherein the pressurizing element includes a compressed foam piece.

7. The system of claim 1, wherein the pressurizing element is configured to expand when heated.

8. The system of claim 1, wherein the breaking element includes a piercing element actuated by a cam, wherein the piercing element is configured to pierce at least a portion of the sealed reservoir.

9. The system of claim 8, wherein the cam is configured to be rotated by a shape-memory alloy wire.

10. The system of claim 8, wherein the piercing element in configured to pierce a seal of the sealed reservoir in contact with the transdermal membrane.

11. The system of claim 8, wherein the cam includes a small end and a large end and the piercing element is coupled to the large end and a shape-memory alloy is coupled to the small end.

12. A method for delivering bioactive agents comprising:
placing a drug cartridge (DC) in contact with a skin of a patient, the DC comprising one or more sealed reservoirs containing a drug formulation and one or more outlet ports configured to guide the drug formulation onto a transdermal membrane;
pressurizing, by a pressurizing element, a first side of at least one of the one or more sealed reservoirs; and
actuating a breaking element and breaking a second side opposite the first side of the at least one of the one or more sealed reservoirs with the breaking element forcing the drug formulation out of the at least one of the one or more sealed reservoirs.

13. The method of claim 12, wherein actuating the breaking element includes heating the breaking element to pierce the at least one of the one or more sealed reservoirs.

14. The method of claim 12, wherein the breaking element comprises a shape-memory allow wire.

15. The method of claim 12, wherein actuating the breaking element comprises passing a current through a wire, extending the wire to pierce that at least one of the one or more sealed reservoirs.

16. The method of claim 12, wherein the pressurizing element provides a constant pressure on the at least one of the one or more sealed reservoirs.

17. The method of claim 12, wherein actuating the breaking element comprises rotating a cam and piercing the at least one of the one or more sealed reservoirs with a piercing element coupled to the cam.
